SEQUENCE LISTING (89 sequences)

SEQ ID NO: 1 - DNA, 225 bp, Artificial sequence
  cDNA sequence of the endogenous protein M of WNV IS98-ST1
tcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagc

SEQ ID NO: 2 - protein, 75 aa, Artificial sequence
  Endogenous protein M of WNV IS98-ST1
SLTVQTHGESTLANKKGAWMDSTKATRYLVKTESWILRNPGYALVAAVIGWMLGSNTMQRVVFVVLLLLVAPAYS

SEQ ID NO: 3 - DNA, 225 bp, Artificial sequence
  cDNA sequence coding for a mutated WNV M protein
  misc_feature (106)..(108): n is a, c, g, or t; nnn is a codon coding either for phenylalanine (TTT or TTC), or tryptophan (TGG), or tyrosine (TAA, TAG or TGA)
  misc_feature (127)..(129): n is a, c, g, or t; nnn is a codon coding for glycine (GGA, GGT, GGC or GGG)
tcactgacagtgcagacacacggagaaagcactctagcgaacaagaagggggcttggatggacagcaccaaggccacaaggtatttggtaaaaacagaatcatggnnnttgaggaaccctggatatnnnctggtggcagccgtcattggttggatgcttgggagcaacaccatgcagagagttgtgtttgtcgtgctattgcttttggtggccccagcttacagc

SEQ ID NO: 4 - protein, 75 aa, Artificial sequence
  mutated WNV M protein
  misc_feature (36)..(36): Xaa is Phe or Trp or Tyr
SLTVQTHGESTLANKKGAWMDSTKATRYLVKTESWXLRNPGYGLVAAVIGWMLGSNTMQRVVFVVLLLLVAPAYS

SEQ ID NO: 5 - DNA, 225 bp, Artificial sequence
  cDNA sequence coding for a mutated WNV M protein
tcactgacagtgcagacacacggagaaagcactctagcgaacaagaagggggcttggatggacagcaccaaggccacaaggtatttggtaaaaacagaatcatggttcttgaggaaccctggatatggactggtggcagccgtcattggttggatgcttgggagcaacaccatgcagagagttgtgtttgtcgtgctattgcttttggtggccccagcttacagc

SEQ ID NO: 6 - protein, 75 aa, Artificial sequence
  mutated WNV M protein
SLTVQTHGESTLANKKGAWMDSTKATRYLVKTESWFLRNPGYGLVAAVIGWMLGSNTMQRVVFVVLLLLVAPAYS

SEQ ID NO: 7 - protein, 3433 aa, Artificial sequence
  WN IS98-ST1 polyprotein, wherein protein M is I36F and A43G mutated
MSKKPGGPGKSRAVNMLKRGMPRVLSLIGLKRAMLSLIDGKGPIRFVLALLAFFRFTAIAPTRAVLDRWRGVNKQTAMKHLLSFKKELGTLTSAINRRSSKQKKRGGKTGIAVMIGLIASVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWFLRNPGYGLVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHADTGCAIDISRQELRCGNGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNAYNADMIDPFQLGLLVVFLATQEVLRKRWTAKISMPAILIALLVLVFGGITYTDVLRYVILVGAAFAESNSGGDVVHLALMATFKIQPVFMVASFLKARWTNQENILLMLAAVFFQMAYHDARQILLWEIPDVLNSLAVAWMILRAITFTTTSNVVVPLLALLTPRLRCLNLDVYRILLLMVGIGSLIREKRSAAAKKKGASLLCLALASTGLFNPMILAAGLIACDPNRKRGWPATEVMTAVGLMFAIVGGLAELDIDSMAIPMTIAGLMFAAFVISGKSTDMWIERTADISWESDAEITGSSERVDVRLDDGENFQLMNDPGAPWKIWMLRMVCLAISAYTPWAILPSVVGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQITVLDLHPGAGKTRRILPQIIKEAINRRLRTAVLAPTRVVAAEMAEALRGLPIRYQTSAVPREHNGNEIVDVMCHATLTHRLMSPHRVPNYNLFVMDEAHFTDPASIAARGYISTKVELGEAAAIFMTATPPGTSDPFPESNSPISDLQTEIPDRAWNSGYEWITEYTGKTVWFVPSVKMGNEIALCLQRAGKKVVQLNRKSYETEYPKCKNDDWDFVITTDISEMGANFKASRVIDSRKSVKPTIITEGEGRVILGEPSAVTAASAAQRRGRIGRNPSQVGDEYCYGGHTNEDDSNFAHWTEARIMPDNINMPNGLIAQFYQPEREKVYTMEGEYRLRGEERKNFLELLRTADLPVWLAYKVAAAGVSYHDRRWCFDGPRTNTILEDNNEVEVITKLGERKILRPRWIDARVYSDHQALKAFKDFASGKRSQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATAWSLYAVTTAVLTPLLKHLITSDYINTSLTSINVQASALFTLARGFPFVDVGVSALLLAAGCWGQVTLTVTVTAATLLFCHYAYMVPGWQAEAMRSAQRRTAAGIMKNAVVDGIVATDVPELERTTPIMQKKVGQIMLILVSLAAVVVNPSVKTVREAGILITAAAVTLWENGASSVWNATTAIGLCHIMRGGWLSCLSITWTLIKNMEKPGLKRGGAKGRTLGEVWKERLNQMTKEEFTRYRKEAIIEVDRSAAKHARKEGNVTGGHSVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKINNRIERLRREYSSTWHHDENHPYRTWNYHGSYDVKPTGSASSLVNGVVRLLSKPWDTITNVTTMAMTDTTPFGQQRVFKEKVDTKAPEPPEGAKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGTRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVIGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLIGTRARATWAENIQVAINQVRAIIGDEKYVDYMSSLKRYEDTTLVEDTVL

SEQ ID NO: 8 - DNA, 31 bp, Artificial sequence
  Forward primer (M-I36F WNV)
aaaacagaatcatggttcttgaggaaccctg

SEQ ID NO: 9 - DNA, 32 bp, Artificial sequence
  Reverse primer (M-I36F WNV)
ccagggttcctcaagaaccatgattctgtttt

SEQ ID NO: 10 - DNA, 25 bp, Artificial sequence
  Forward primer (M-A43G WNV)
accctggatatggactggtggcagc

SEQ ID NO: 11 - DNA, 25 bp, Artificial sequence
  Reverse primer (M-A43G WNV)
gctgccaccagtccatatccagggt

SEQ ID NO: 12 - DNA, 21 bp, Artificial sequence
  Primer
gcggcaatattcatgacagcc

SEQ ID NO: 13 - DNA, 21 bp, Artificial sequence
  Primer
cgggatctcagtctgtaagtc

SEQ ID NO: 14 - DNA, 20 bp, Artificial sequence
  Primer
ggtcggagtcaacggatttg

SEQ ID NO: 15 - DNA, 20 bp, Artificial sequence
  Primer
actccacgacgtactcagcg

SEQ ID NO: 16 - protein, 49 aa, Artificial sequence
  ectoM and part of TM1 of Dengue Virus 1 (DV1)
SVALAPHVGLGLETRTETWMSSEGAWKQIQKVETWALRHPGFTVIALFL

SEQ ID NO: 17 - protein, 49 aa, Artificial sequence
  ectoM and part of TM1 of Dengue Virus 2 (DV2)
SVALVPHVGMGLETRTETWMSSEGAWKHAQRIETWILRHPGFTIMAAIL

SEQ ID NO: 18 - protein, 49 aa, Artificial sequence
  ectoM and part oof TM1 f Dengue Virus 3 (DV3)
SVALAPHVGMGLDTRAQTWMSAEGAWRQVEKVETWAFRHPGFTILALFL

SEQ ID NO: 19 - protein, 75 aa, Artificial sequence
  endogenous M protein of Dengue Virus 4 (DV4)
SVALTPHSGMGLETRAETWMSSEGAWKHAQRVESWILRNPGFALLAGFMAYMIGQTGIQRTVFFVLMMLVAPSYG

SEQ ID NO: 20 - protein, 75 aa, Artificial sequence
  endogenous M protein of Japanese Encephalitis Virus (JEV)
SVSVQTHGESSLVNKKEAWLDSTKATRYLMKTENWIIRNPGYAFLAAVLGWMLGSNNGQRVVFTILLLLVAPAYS

SEQ ID NO: 21 - protein, 75 aa, Artificial sequence; identical to SEQ ID NO: 2
  truncated endogenous M protein of West Nile Virus (WNV)

SEQ ID NO: 22 - protein, 75 aa, Artificial sequence
  endogenous M protein of Zika Virus (ZIKV), PF-13 strain
AVTLPSHSTRKLQTRSQTWLESREYTKHLIRVENWIFRNPGLALAAAAIAWLLGSSTSQKVIYLVMILLIAPAYS

SEQ ID NO: 23 - protein, 49 aa, Artificial sequence
  ectoM and part of TM1 of Yellow Fever Virus (YFV)
AIDLPTHENHGLKTRQEKWMTGRMGERQLQKIERWLVRNPFFAVTALTI

SEQ ID NO: 24 - protein, 49 aa, Artificial sequence
  ectoM and part of TM1 of Yellow Fever Virus-17D vaccine strain (YFV-17D)
AIDLPTHENHGLKTRQEKWMTGRMGERQLQKIERWFVRNPFFAVTALTI

SEQ ID NO: 25 - protein, 49 aa, Artificial sequence; identical to SEQ ID NO: 24
  ectoM and part of TM1 of Yellow Fever Virus-French Neurotropic Virus (YFV-FNV)

SEQ ID NO: 26 - protein, 49 aa, Artificial sequence
  ectoM and part of TM1 of Dengue Virus 4 (DV4)
SVALTPHSGMGLETRAETWMSSEGAWKHAQRVESWILRNPGFALLAGFM

SEQ ID NO: 27 - protein, 49 aa, Artificial sequence
  ectoM and part of TM1 of Japanese Encephalitis Virus (JEV)
SVSVQTHGESSLVNKKEAWLDSTKATRYLMKTENWIIRNPGYAFLAAVL

SEQ ID NO: 28 - protein, 49 aa, Artificial sequence
  ectoM and part of TM1 of West Nile Virus (WNV)
SLTVQTHGESTLANKKGAWMDSTKATRYLVKTESWILRNPGYALVAAVI

SEQ ID NO: 29 - protein, 49 aa, Artificial sequence
  ectoM and part of TM1 of Zika Virus (ZIKV)
AVTLPSHSTRKLQTRSQTWLESREYTKHLIRVENWIFRNPGLALAAAAI

SEQ ID NO: 30 - protein, 43 aa, Artificial sequence
  ectoM and part of TM1 of WNV (from 1 to 43)
SLTVQTHGESTLANKKGAWLDSTKATRYLVKTESWILRNPGFA

SEQ ID NO: 31 - protein, 60 aa, Artificial sequence
  M protein of WNV
SLTVQTHGESTLANKKGAWMDSTKATRYLVKTESWILRNPGYALVAAVIGWMLGSNTMQR

SEQ ID NO: 32 - protein, 60 aa, Artificial sequence
  M protein of ZIKV
AVTLPSHSTRKLQTRSQTWLESREYTKHLIKVENWIFRNPGFALVAVAIAWLLGSSTSQK

SEQ ID NO: 33 - DNA, 28 bp, Artificial sequence
  Forward primer (M-I36F ZIKV)
ggttgaaaactggtttttcaggaacccc

SEQ ID NO: 34 - DNA, 28 bp, Artificial sequence
  Reverse primer (M-I36F ZIKV)
ggggttcctgaaaaaccagttttcaacc

SEQ ID NO: 35 - DNA, 27 bp, Artificial sequence
  Forward primer (M-A43G ZIKV)
aaccccgggtttggactagtggccgtt

SEQ ID NO: 36 - DNA, 26 bp, Artificial sequence
  Reverse primer (M-A43G ZIKV)
aacgccactagtccaaacccggggtt

SEQ ID NO: 37 - DNA, 20 bp, Artificial sequence
  Primer
atggaagacggctgtggaag

SEQ ID NO: 38 - DNA, 20 bp, Artificial sequence
  Primer
gctcccaaccacatgtacca

SEQ ID NO: 39 - DNA, 22 bp, Artificial sequence
  Forward primer
ctgtgtgagctgacaaacttag

SEQ ID NO: 40 - DNA, 20 bp, Artificial sequence
  Forward primer
gacggtaaatgctactgacg

SEQ ID NO: 41 - DNA, 19 bp, Artificial sequence
  Forward primer
ttggaaggagtgtctggag

SEQ ID NO: 42 - DNA, 21 bp, Artificial sequence
  Forward primer
aaagcttggagaatatggaga

SEQ ID NO: 43 - DNA, 18 bp, Artificial sequence
  Forward primer
cattgaacgacctaacgc

SEQ ID NO: 44 - DNA, 20 bp, Artificial sequence
  Forward primer
caagagctgagatgtggaag

SEQ ID NO: 45 - DNA, 22 bp, Artificial sequence
  Forward primer
gaatgtgactcgaagatcattg

SEQ ID NO: 46 - DNA, 18 bp, Artificial sequence
  Forward primer
cctcgtgcagtcacaagt

SEQ ID NO: 47 - DNA, 18 bp, Artificial sequence
  Forward primer
ctagccctgctaacaccc

SEQ ID NO: 48 - DNA, 20 bp, Artificial sequence
  Forward primer
ttggaagatatggatgctca

SEQ ID NO: 49 - DNA, 18 bp, Artificial sequence
  Forward primer
ccactggaacatcaggct

SEQ ID NO: 50 - DNA, 21 bp, Artificial sequence
  Forward primer
gcaagaggttacatttccaca

SEQ ID NO: 51 - DNA, 18 bp, Artificial sequence
  Forward primer
agaaatccgtcgcaagtt

SEQ ID NO: 52 - DNA, 18 bp, Artificial sequence
  Forward primer
tgcctgagcacttcatgg

SEQ ID NO: 53 - DNA, 19 bp, Artificial sequence
  Forward primer
caacagcctggtcactgta

SEQ ID NO: 54 - DNA, 18 bp, Artificial sequence
  Forward primer
acgagaagccggaatttt

SEQ ID NO: 55 - DNA, 18 bp, Artificial sequence
  Forward primer
gtcccggacatgaagagc

SEQ ID NO: 56 - DNA, 18 bp, Artificial sequence
  Forward primer
ggaaaacccctgctcaac

SEQ ID NO: 57 - DNA, 18 bp, Artificial sequence
  Forward primer
ggggggaatgtcacactt

SEQ ID NO: 58 - DNA, 18 bp, Artificial sequence
  Forward primer
ttgtcacctacgccctaa

SEQ ID NO: 59 - DNA, 18 bp, Artificial sequence
  Forward primer
aagagacctgcggctcat

SEQ ID NO: 60 - DNA, 18 bp, Artificial sequence
  Forward primer
aaagtcaggccgggaagt

SEQ ID NO: 61 - DNA, 18 bp, Artificial sequence
  Reverse primer
caagcccccttcttgttc

SEQ ID NO: 62 - DNA, 19 bp, Artificial sequence
  Reverse primer
ttcctttgccaaatagtcc

SEQ ID NO: 63 - DNA, 20 bp, Artificial sequence
  Reverse primer
agtgttgcttgaaaattcca

SEQ ID NO: 64 - DNA, 18 bp, Artificial sequence
  Reverse primer
atggacagccttcccaac

SEQ ID NO: 65 - DNA, 18 bp, Artificial sequence
  Reverse primer
gtgaggcgtttaggtgct

SEQ ID NO: 66 - DNA, 18 bp, Artificial sequence
  Reverse primer
agtcaatctctacccggc

SEQ ID NO: 67 - DNA, 23 bp, Artificial sequence
  Reverse primer
ccaccataaacactggttgtatc

SEQ ID NO: 68 - DNA, 18 bp, Artificial sequence
  Reverse primer
caatgtcaagctctgcca

SEQ ID NO: 69 - DNA, 18 bp, Artificial sequence
  Reverse primer
cctctccgctcatcaaag

SEQ ID NO: 70 - DNA, 18 bp, Artificial sequence
  Reverse primer
acaaccctggttggtgct

SEQ ID NO: 71 - DNA, 18 bp, Artificial sequence
  Reverse primer
ttgggtactccgtctcgt

SEQ ID NO: 72 - DNA, 18 bp, Artificial sequence
  Reverse primer
cacctccggtcgtggtat

SEQ ID NO: 73 - DNA, 18 bp, Artificial sequence
  Reverse primer
gagagcagcaacattccg

SEQ ID NO: 74 - DNA, 18 bp, Artificial sequence
  Reverse primer
aaccgggaaccatgtagg

SEQ ID NO: 75 - DNA, 18 bp, Artificial sequence
  Reverse primer
agcgatcgacttcgatga

SEQ ID NO: 76 - DNA, 21 bp, Artificial sequence
  Reverse primer
ggagcagctccatcttctcta

SEQ ID NO: 77 - DNA, 19 bp, Artificial sequence
  Reverse primer
catggttttgagaggagcc

SEQ ID NO: 78 - DNA, 18 bp, Artificial sequence
  Reverse primer
ccagccagctgtgtcatc

SEQ ID NO: 79 - DNA, 19 bp, Artificial sequence
  Reverse primer
tcataccatccagttgacg

SEQ ID NO: 80 - DNA, 19 bp, Artificial sequence
  Reverse primer
ggttgatagccacctggat

SEQ ID NO: 81 - DNA, 18 bp, Artificial sequence
  Reverse primer
tttcgccctggttaacac

SEQ ID NO: 82 - DNA, 18 bp, Artificial sequence
  Reverse primer
atcctgtgttctcgcacc

SEQ ID NO: 83 - protein, 75 aa, Artificial sequence
  mutated ZIKV M protein, MR766 strain
  misc_feature (36)..(36): Xaa is Phe or Trp or Tyr
AVTLPSHSTRKLQTRSQTWLESREYTKHLIKVENWXFRNPGFGLVAVAIAWLLGSSTSQKVIYLVMILLIAPAYS

SEQ ID NO: 84 - protein, 75 aa, Artificial sequence
  endogenous M protein of Zika Virus (ZIKV), MR766 strain
AVTLPSHSTRKLQTRSQTWLESREYTKHLIKVENWIFRNPGFALVAVAIAWLLGSSTSQKVIYLVMILLIAPAYS

SEQ ID NO: 85 - DNA, 225 bp, Artificial sequence
  cDNA sequence of the endogenous protein M of USUV bologna 2009 strain
tcgatcgcagtgcagacgcacggggagagtatgctggctaacaagaaggatgcttggctagactcaaccaaggcttcgagatacctgatgaagactgagaattggattatcaggaatcctgggtatgcttttgtagctgtcctcttgggctggatgctgggaagcaacaatggacaaagggtcgttttcgtcgttctcttacttctcgtggcgcctgcttatagc

SEQ ID NO: 86 - protein, 75 aa, Artificial sequence
  Endogenous protein M of USUV bologna 2009 strain
SIAVQTHGESMLANKKDAWLDSTKASRYLMKTENWIIRNPGYAFVAVLLGWMLGSNNGQRVVFVVLLLLVAPAYS

SEQ ID NO: 87 - protein, 75 aa, Artificial sequence
  mutated USUV M protein, bologna 2009 strain
  misc_feature (36)..(36): Xaa is Phe or Trp or Tyr
SIAVQTHGESMLANKKDAWLDSTKASRYLMKTENWXIRNPGYGFVAVLLGWMLGSNNGQRVVFVVLLLLVAPAYS

SEQ ID NO: 88 - DNA, 223 bp, Artificial sequence
  cDNA sequence of the endogenous M protein of Zika Virus (ZIKV), MR766 strain
ccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacag

SEQ ID NO: 89 - DNA, 223 bp, Artificial sequence
  cDNA sequence coding for a mutated ZIKV MR766 strain M protein
  misc_feature (105)..(107): n is a, c, g, or t; nnn is a codon coding either for phenylalanine (TTT or TTC), or tryptophan (TGG), or tyrosine (TAA, TAG or TGA)
  misc_feature (126)..(128): n is a, c, g, or t; nnn is a codon coding for glycine (GGA, GGT, GGC or GGG)
ccgtgacgctcccttctcactctacaaggaagttgcaaacgcggtcgcagacctggttagaatcaagagaatacacgaagcacttgatcaaggttgaaaactggnnnttcaggaaccccgggtttnnnctagtggccgttgccattgcctggcttttgggaagctcgacgagccaaaaagtcatatacttggtcatgatactgctgattgccccggcatacag

(19) United States
(12) Patent Application Publication
(10) Pub. No.: US 2023/0167415 A1
(43) Pub. Date: Jun. 1, 2023

(54) A LIVE AND ATTENUATED FLAVIVIRUS COMPRISING A MUTATED M PROTEIN

(71) Applicants: INSTITUT PASTEUR, Paris Cedex 15 (FR); UNIVERSITE DE PARIS, Paris (FR); CENTRE NATIONAL DE LA RECHERCHE SCIENTIFIQUE, Paris Cedex 16 (FR)

(72) Inventors: Nathalie PARDIGON, Sèvres (FR); Justine BASSET, Paris (FR); Félix Augusto REY, Gif sur Yvette (FR)

(21) Appl. No.: 17/593,446

(22) PCT Filed: Mar. 27, 2020
(86) PCT No.: PCT/IB2020/000302

§ 371 Date: Sep. 19, 2021

Related U.S. Application Data
(60) Provisional application No. 62/825,734, filed on Mar. 28, 2019.

(51) Int. Cl. C12N 7/00; C07K 14/005

(52) U.S. Cl. CPC C12N 7/00; C07K 14/005; C12N 2770/24134; C12N 2770/24122; C12N 2770/24171

(57) ABSTRACT

The application relates to the attenuation of a flavivirus, such as a West Nile Virus (WNV), Zika virus (ZIKV), Japanese Encephalitis Virus (JEV), Dengue Virus (DV), in particular DV4, or Usutu virus. The application notably provides a live and attenuated flavivirus, such as a live and attenuated WNV or ZIKV, comprising a mutated M protein. Said mutated M protein comprises or consists of a sequence, wherein at least the amino acids at positions 36 and 43 in said sequence are mutated. The application also provides embodiments deriving from said live and attenuated flavivirus, such as a WNV or ZIKV, such as nucleic acids, cells, cDNA clones, immunogenic compositions as well as uses and methods.

### FIELD OF THE INVENTION

The application relates to the attenuation of flaviviruses, such as West Nile Virus (WNV) or Zika virus (ZIKV). The application notably provides a live and attenuated flavivirus, such as a WNV or ZIKV, comprising a mutated M protein. Said mutated M protein comprises or consists of a sequence, wherein the amino acids at position 36 in the ectodomain and position 43 in the transmembrane domain 1 in said sequence are mutated. The application also provides additional embodiments deriving from said live and attenuated flavivirus, such as a WNV or ZIKV, such as nucleic acids, cDNA clones, immunogenic compositions as well as uses and methods.

### BACKGROUND OF THE INVENTION

Flaviviruses such as West Nile Virus (WNV), Zika virus (ZIKV), Usutu virus (USUV), Japanese Encephalitis Virus (JEV), Dengue Virus (DV) and Yellow Fever Virus (YFV) viruses, are arthropod-borne pathogens (arboviruses) that are transmitted through the bite of an infected mosquito and may cause serious human diseases worldwide (Lindenbach B D et al, Adv Virus Research, 2003, 59, 23-61). To date, very few vaccines against flaviviruses are commercially available. The first one was the live-attenuated vaccine 17D against YFV (Barrett, ADT Yellow Fever Vaccines Biologicals 1997). There are also live-attenuated and inactivated vaccines against JEV such as the live-attenuated virus vaccine SA₁₄-14-2 (Yun S I, Lee Y M. Hum Vaccin Immunother. 2014 February, 10(2): 263-279) and inactivated vaccines against tick-borne encephalitis virus (Lani R et al, Ticks Tick Borne Dis. 2014 Sep., 5(5): 457-465). Determination of the attenuation factors of these viruses can help in the development of new molecular vaccines. Among the different proteins encoded by the virus genome, it seems that structural proteins (capsid C, membrane M and envelope E) have a role in the pathogenesis of flaviviruses (Kofler R M et al, J Virol. 2002 April, 76(7): 3534-3543; Zhu W et al, Virus Res. 2007 June; 126(1-2): 226-232; Keelapang et al, Vaccine, 2013, 31 (44), 5134-5140; Langevin S A et al, J Gen Virol. 2011, 92(Pt 12): 2810-2820; Yun et al, PLoS Pathogens 2014, 10(7): e1004290; Yang D. et al, Vaccine, 2014, 32(23): 2675-2681; Guirakhoo F et al, J Virol. 2004, 78(18): 9998-10008; Arroyo J. et al, J_Virol. 2001, 75(2): 934-942; Zhao Z. et al, J Gen Virol. 2005, 86(8): 2209-2220; Mandl et al, J Virol. 2000, 74(20): 9601-9609; Holzmann H. et al, J Virol. 1990, 64(10): 5156-5159; Lee E. et al, J Virol 2008, 82(12): 6024-6033).

WNV contains a positive single-stranded RNA genome encoding a single polyprotein that is processed into three structural proteins, the capsid (C), the precursor of membrane (prM) and the envelope (E) proteins, and seven nonstructural proteins (NS1, NS2A, NS2B, NS3, NS4A, NS4B and NS5).

The membrane protein is synthesized as a precursor prM. It is cleaved in the trans-Golgi apparatus during viral particles secretion into pr and M (Li L et al Science. 2008, 319(5871):1830-1834). This cleavage is mandatory to produce infectious particles (Randolph V B et al, Virology, 1990, 174(2): 450-458). The resulting M protein is composed of an ectodomain (ectoM) consisting of 40 amino acids and 2 transmembrane domains TM1 and 2 of 35 amino acids (Zhang et al, EMBO J. 2003, 22(11): 2604-2613).

To date, little is known about the role of protein M. It has been shown that prM protein acts as a chaperone for the E protein folding (Konishi et al, J Virol. 1993, 67(3): 1672-1675) and prevents fusion within the infected cells (Yu et al, J Virol. 2009, 83(23): 12101-12107). It has also been disclosed that the C-terminal helical domain of DENV ectoM is involved in virus assembly (Pryor et al, J Gen Virol, 2004, 85(Pt 12): 3627-3636; Hsieh et al, J Virol. 2010, 84(9): 4782-4797) and entry (Hsieh et a, J Virol. 2010, 84(9): 4782-4797), and that DENV, YFV strain Asibi and WNV ectoM induces apoptosis in mammalian cells. In this study, it has been shown that a mutation of the leucine at position 36 of YFV ectoM into a phenylalanine or a mutation of the isoleucine at position 36 of DENV ectoM into a phenylalanine reduces the induction of apoptosis (Catteau et al, J. Gen Virol. 2003, 84(10): 2781-2793; U.S. Pat. No. 7,785,604 patent). In particular, patent U.S. Pat. No. 7,785,604 describes that a nonapeptide (ApoptoM) from flavivirus ectoM is able to modulate specifically the apoptotic activity of diverse flaviviruses, and that the proapoptotic properties of ectoM are conserved among apoptosis-inducing flaviviruses, i.e. WNV, JEV, DV and YFV. Moreover, the interaction of the M protein of WNV with a light chain of human dynein has been shown to play a role in virus replication (Brault et al, 2011, Virology, 417(2): 369-378).

McElroy et al. have demonstrated that the replacement of the leucine at position 36 of YFV strain Asibi ectoM into a phenylalanine (YFV-17D vaccine strain) reduces the mean dissemination of YFV in mosquitoes. A higher mean dissemination was obtained when the sequences encoding the full M-E proteins or the E protein domain III of YFV-17D vaccine strain were incorporated to replace the same proteins of YFV strain Asibi (McElroy et al, J. Gen Virol., 2006, 87, 2993-3001). More recently, de Wispelaere et al showed that the replacement of the amino acid at position 36 in the M protein of JEV (which is an isoleucine) by the amino acid phenylalanine, leads to attenuation (de Wispelaere et al, J Virol. 2016, 90(5): 2676-2689).

### SUMMARY OF THE INVENTION

The application provides a live and attenuated flavivirus, such as a WNV or a ZIKV, comprising a mutated M protein. Said mutated M protein comprises or consists of a sequence, wherein at least the amino acids at position 36 and 43 in said sequence are mutated, more particularly replaced by another amino acid, more particularly the amino acid phenylalanine (F), tryptophan (W) or tyrosine (Y) at position 36, more particularly by the amino acid phenylalanine (F), and the amino acid glycine (G) at position 43.

The application also provides means deriving from said live and attenuated flavivirus, such as nucleic acids, more particularly RNA and cDNA, proteins and polypeptides, more particularly recombinant cDNA clones as well as immunogenic compositions and vaccines.

The application also provides as uses and methods, more particularly uses and methods to prevent a flavivirus infection, such a WNV infection or a Zika infection, in a mammalian host, especially in a human or an animal host (in particular for WNV or USUV).

### BRIEF DESCRIPTION OF THE FIGURES

FIG. 1: WNV IS98 two-plasmid infectious clone technique. Viral production using the two-plasmids infectious clone method is represented.

FIGS. 2A and 2B: WNV M protein structural analysis and M-I36F mutation. FIG. 2A: Side view of E and M heterodimers (PDB 5wsn). Isoleucine 36, located in the ectodomain of M protein, is represented as stick. FIG. 2B: Mutated phenylalanine in position 36 (Phe 36) causes negative interactions with the side chain of alanine 43 (Ala 43).

FIG. 3: M-I36F and M-I36F/A43G mutations affected WNV cycle in mammalian cell. FIG. 3A: Viruses ability to attach and penetrate into SK-N-SH cells was analyzed by qRT-PCR at early time point post-infection. FIG. 3B: WNV WT and mutants replication was monitored up to 24 h pi. Amplification of genomic viral RNA was measured by qRT-PCR. FIG. 3C: Viral protein synthesis was investigated by Western Blot. FIG. 3D: Supernatants of SK-N-SH cells infected with WNV WT or mutants collected at 24 h, 48 h and 72 h pi were titrated. FIG. 3E: viral RNA secreted in SK-N-SH supernatants collected at 24 h, 48 h and 72 h were extracted and quantify by qRT-PCR. FIG. 3F: Specific infectivity was calculated as a ration of RNA copies to infectious particles.

FIGS. 4A 4B, 4C, 4D and 4E (A: NI; B: WT; C: A43G; D: I36F/A43G; E: I36F): M protein mutations lead to extensive viral particles retention. M-I36F and M-I36F/A43G mutant particles are retained within the ER lumen of infected mammalian cells but not in mosquito cells. Vero cells were infected with wild-type or mutated WNV in positions M-36 and/or M-43 at a MOI of 10 and examined by transmission electron microscopy at 24 h pi. Transmission electron microscopy of Vero cells infected with either WNV WT (B), M-A43G (C) M-I36F(D), M-I36F/A43G (E) (MOI=10) or uninfected (A) was performed. Examples of viral particles located in the ER lumen are indicated by arrows. Inset bars: 100 nm.

FIG. 5: Mutations in the M protein modify viral particles morphology and lead to defective particles production. FIGS. 5A, 5B, and 5C: Morphology of WT (A), M-A43G (B) and M-I36F/A43G (C) viruses secreted in the supernatants of infected Vero cells were observed using negative staining electron microscopy. FIGS. 5D, 5E and 5F: the specificity of the particles was confirmed by negative staining with uranyl. FIG. 5G: The ability of WNV WT, M-A43G and M-I36F/A43G viruses produced in Vero cells to attach and penetrate into SK-N-SH cells was tested by qRT-PCR at early time points post-infection (upper image) and at 1 h at 4° C. post-infection (lower image). FIG. 5H: The ability of WNV WT, M-A43G and M-I36F/A43G viruses produced in Vero cells to attach to C6/36 cell surface was tested by qRT-PCR at 1 h at 4° C. post-infection.

FIG. 6: Alteration of WNV particles morphology leads to viral attenuation in a mouse model. FIG. 6A: Three-week-old BALB/c female mice were injected intraperitoneally with 50 focus forming units (ffu) of WNV WT virus, or with M-I36F, M-A43G, M-I36F/A43G mutant viruses. Survival percentages were calculated (****: P<0.0001). FIG. 6B: Viremia developed by mice was assessed by qRT-PCR. FIG. 6C: Mice growth was followed every day by measuring their body weight. FIG. 6D: At 28 days post inoculation mice that survived the infection were challenged with a lethal dose of 1000 ffu of WNV WT. Survival percentages were calculated (**: P<0.0036). FIG. 6E: Sera were collected 27 days after inoculation from the mice that survived and were diluted. WNV specific-IgG and neutralizing antibodies were measured by ELISA. Seroneutralization assay was performed on dilutions using WNV WT virus as target.

FIG. 7: Alignment of M protein sequences from flaviviruses. The sequences shown in FIG. 7 are assigned the following sequence identification numbers: Dengue Virus 1 (DV1) (SEQ ID NO: 16), Dengue Virus 2 (DV2) (SEQ ID NO: 17), Dengue Virus 3 (DV3) (SEQ ID NO: 18), Dengue Virus 4 (DV4) (SEQ ID NO: 26), Japanese Encephalitis Virus (JEV) (SEQ ID NO: 27), West Nile Virus (WNV) (SEQ ID NO: 28), Zika Virus (ZIKV) (SEQ ID NO: 29), Yellow Fever Virus (YFV) (SEQ ID NO: 23), Yellow Fever Virus-17D vaccine strain (17D) (SEQ ID NO: 24), and Yellow Fever Virus-French Neurotropic Virus (FNV) vaccine strain (SEQ ID NO: 25).

FIG. 8: The nature of M-36 residue impacts WNV infectious cycle by potentially disrupting the M protein 3-dimensional structure.

(A): WNV membrane protein precursor (prM) organization showing ectodomain (ectoM) and part of transmembrane domain 1 (TM1) sequences (SEQ ID NO: 30). (B): Viral stocks were collected from C6/36 cell supernatants at times indicated and titrated by foci-forming assay (FFA) in Vero cells. No statistical difference was observed. (C): Foci morphology of wild-type WNV, M-I36F and M-I36A mutated viral stocks collected from C6/36 supernatants, observed on Vero cells. Vero cells were infected with the indicated virus and foci were observed 48 h pi. (D): Growth curves of wild-type, M-I36F and M-I36A mutant WNV. SK-N-SH cells were infected with the indicated virus at a MOI of 1, cell supernatants were collected at indicated times for quantitation of virus titers by FFA using Vero cells. (E): Structure of M-E mature heterodimers (PDB accession number 5wsn). The insert zooms into the A43-F36 contact. The F36 aromatic ring clashes with the side chain of the A43 located in the TMD-1. (F): Same as (E) with alanine at position M-36. The insert zooms into the A36-A43 contact. No clash between A36 and A43 was observed. The image was generated using PyMOL. The data are representative of 3 independent experiments and error bars indicate standard deviation (SD). *p-value<0.05; **p-value<0.01, ***p-value<0.001.

FIG. 9: Phenotypical characterization of WNV M-I36F and/or M-A43G mutants effect on WNV replication in vitro.

(A, B): Viral stocks of WNV wild-type and mutants M-A43G, M-I36F and M-I36F/A43G were used at a MOI of 1 to infect (A): Vero cells or (B): C6/36 cells. At the indicated time points, cells were harvested and levels of WNV genomic RNA were quantified by RT-qPCR. (C, D, E, F): Growth curves and genome quantitation of wild-type, M-I36F, M-A43G and M-I36F/A43G mutated WNV produced in Vero cells. Vero (C, E) and C6/36 cells (D, F) were infected with the indicated viruses at a MOI of 1, cell supernatants were collected at indicated times for quantitation of virus titers by FFA using Vero cells (C, D) or genome quantitation by RT-qPCR (E, F). (G, H): Cell viability. Vero (G) or SK-N-SH (H) cells were infected with the indicated viruses at a MOI of 1, cells were harvested at indicated times, cell viability was evaluated using CellTiter Glo and represented as a percentage of non-infected control cells. The data are representative of 3 independent experiments and error bars indicate standard deviation (SD). *p-value<0.05; **p-value<0.01, ***p-value<0.001.

FIG. 10. M-I36F mutation effects on WNV antigenic profile.

(A, B): Wild-type and mutated WNV surface epitope exhibition was analyzed by direct ELISA. 200 ng of different UV-inactivated viruses collected from C6/36 cells (A) or Vero cells (B) were coated and tested with increasing concentrations of mAb 4G2. (C, D): Same as (A) and (B) using indirect non-competitive ELISA. (E, F): Same as (A) and (B) but with increasing concentrations of polyclonal anti-WNV antibodies. (G, H): Infectious capacity of mutant virus M-I36F/A43G is impaired when the virus is produced in mammalian cells. SK-N-SH and C6/36 cells were placed at 4° C. for 1 h, then infected at a MOI (amount of viral genomic RNA) of 10 for 1 h at 4° C. with the indicated viruses. (G): SK-N-SH cells were collected and viral genomes attached to the cell-surface were quantified by RT-qPCR. (H): Same as (G) with C6/36 cells. (I): Levels of E, immature prM and mature M glycoproteins were tested under denaturing conditions by Western Blot using a polyclonal anti-WNV antibody. The same amount of viral RNA was loaded in each well. The histograms indicate the median value and the interquartile range determined from triplicate of three independent experiments. *p-value<0.05; **p-value<0.01; ***p-value<0.001.

FIG. 11. Combined M-I36F and M-A43G mutations highly attenuate WNV and elicit WNV-specific humoral response in a mouse model.

(A): Survival curves of 3-weeks-old BALB/c mice inoculated with 50 FFU of the indicated viruses by i.p. route. (B): Mice growth curve. Mice weight was measured every day pi and is represented as a percentage of the starting body weight. (C): Viral load in mice blood. Viral RNA loads were quantified by RT-qPCR. Dotted line indicates detection limit. (D, E): WNV specific-IgG and neutralizing antibodies were measured by ELISA and PRNT₅₀ respectively. (F): Survivor mice were challenged with 1000 FFU of wild-type WNV at day 28 pi. Mice were monitored for clinical symptoms and mortality for 25 days. The data are representative of at least two independent experiments and error bars indicate the SD. (*p-value<0.05; **p-value<0.01, ***p-value<0.001).

FIG. 12. M-I36F and/or M-A43G mutation do not alter WNV secretion from infected C6/36 mosquito cells.

C6/36 cells were infected with wild-type WNV or mutated at position M-36 and/or M-43 at a MOI of 10 and examined by transmission electron microscopy at 24 h pi. (A): C6/36 cell infected with WNV WT. (B): same with mutated virus M-A43G. (C): same with mutated virus M-I36F. (D): Same with double mutant virus M-I36F/A43G. (E): Uninfected C6/36 cell. Examples of viral particles located in the ER lumen are indicated by arrows.

FIG. 13. Secreted mutant virions M-I36F/A43G display an altered morphology.

Wild-type and mutated viral particles collected from supernatants of Vero cells infected at a MOI of 10 for 24 h, were concentrated and purified. (A, B, C): Particles were stained negatively with uranyl and observed by transmission electron microscopy. (A): WNV WT particles. (B): WNV M-A43G particles. (C): WNV M-I36F/A43G particles. (D, E, F): Viral particles were labeled by immunogold with an anti-protein E pan-flavivirus antibody (mAb 4G2) and observed by transmission electron microscopy. (D): WNV WT particles. (E): WNV M-A43G particles. (F): WNV M-I36F/A43G particles. Bars=100 nm.

FIG. 14. M-I36F and/or M-A43G mutation do not alter WNV morphology when produced in C6/36 mosquito cells.

Wild-type and mutated viral particles collected from supernatants of C6/36 cells infected at a MOI of 10 for 24 h, were concentrated and purified. (A, B, C, D): Particles were stained negatively with uranyl and observed by transmission electron microscopy. (A): WNV WT particles. (B): WNV M-A43G particles. (C): WNV M-I36F particles. (D): WNV M-I36F/A43G particles. Bars=200 nm.

FIG. 15. M-I36F and/or M-A43G mutation do not impair WNV infectious capacity when produced in insect cells. SK-N-SH and C6/36 cells were placed at 4° C. for 1 h, prior to infection at a MOI of 10 (amount of viral genomic RNA) for 1 h at 4° C. with the indicated viruses. (A): SK-N-SH cells were collected and viral genomes attached to the cell surface were quantified by RT-qPCR. (B): Same as (A) with C6/36 cells. The histograms indicate the median value and the interquartile range determined from triplicates of three independent experiments. Error bars indicate standard SD.

FIG. 16. Sequence comparison between Zika and West Nile virus M protein.

M protein amino acid sequence of West Nile (WNV) (SEQ ID NO: 31) and Zika (ZIKV) (SEQ ID NO: 32) viruses were aligned. Similarity (:) and identity (|) are expressed in number of residues and % (in parenthesis) are indicated.

FIG. 17. M-I36F and M-I36F/A43G mutations affected ZIKV plaque morphology in mammalian cell.

ZIKV is an arbovirus that infects both mosquitoes and mammals. After electroporation, the different viruses were produced in Aedes albopictus C6/36 cells and the stability of each mutation was confirmed by Sanger sequencing (data not shown). Viruses were titrated in Vero cells. The inventors observed differences in plaque morphology, with WT (D) and M-A43G (C) mutant displaying mostly large plaques, while M-I36F (A) and M-I36F/A43G (B) mutants displayed a mix of small and medium size plaques.

FIG. 18. M-I36F and M-I36F/A43G mutations affect ZIKV infectious cycle in mammalian cell.

18A. Supernatants from infected Vero cells at a MOI of 1, were harvested at 24 h and 48 h pi and infectious particles production was quantified. A decrease of around 2.3 logs and 1.9 logs in titers was observed in the supernatants of cells infected with ZIKV M-I36F and M-I36F/A43G respectively as compared to WT. Interestingly, ZIKV M-A43G produced as many infectious particles as WT, showing that the M-A43G mutation alone did not affect WNV cycle. 18B. Supernatants from infected SK-N-SH cells (MOI=1) were harvested at 24 h and 48 h pi and infectious particle production was quantified. A decrease of around 2.1 logs and 1.6 logs in titers was observed in the supernatants of cells infected with ZIKV M-I36F and M-I36F/A43G respectively as compared to WT. As observed in Vero cells, ZIKV M-A43G produced as many infectious particles as WT. 18C. In addition, viral genomic RNA extracted from supernatants of Vero cells was measured by RT-qPCR. Less viral RNA were observed in the supernatants of cells infected with ZIKV M-I36F (2.29 logs) and ZIKV M-I36F/A43G (1.9 logs) indicating that lower numbers of infectious particles were released from Vero cells infected with either ZIKV M-I36F or ZIKV M-I36F/A43G as compared to WT. 18D. Viral genomic RNA extracted from supernatants of SK-N-SH cells and quantified by RT-qPCR also showed less viral RNA in the supernatants of cells infected with ZIKV M-I36F (1 log) and ZIKV M-I36F/A43G (1.2 logs). 18E. Relative specific infectivity of each virus secreted in Vero cell supernatants was measured as a ratio of ZIKV RNA to infectious particles. 18F. Relative specific infectivity of each virus secreted in SK-N-SH cell supernatants was measured as a ratio of ZIKV RNA to infectious particles. Specific infectivity of ZIKV mutant viruses was overall similar than that of WT. Altogether, these results strongly suggest that M-I36F/A43G mutations together might alter viral assembly and/or secretion.

FIG. 19. M-I36F and M-I36F/A43G mutations do not affect ZIKV infectious cycle in mosquito cells.

19A. Supernatants from infected C6/36 cells (MOI=1) were harvested at 24 h and 48 h pi and infectious particles production was quantified. No significant difference in titers was observed in the supernatants of cells infected with either ZIKV M-I36F, ZIKV M-I36F/A43G, ZIKV M-A43G or ZIKV WT. 19B. Viral genomic RNA extracted from supernatants of C6/36 cells and quantified by RT-qPCR showed no significant difference in the amount of viral RNA in the supernatants of cells infected either with ZIKV M-I36F, ZIKV M-I36F/A43G, ZIKV M-A43G or ZIKV WT.

### DETAILED DESCRIPTION OF THE INVENTION

The inventors introduced two point mutations into the M protein of a WNV plasmid construct that encodes the structural region of WNV genome, and showed that infection of mammalian cells with mutated WNV particles resulted in a reduced number of secreted viral particles relative to the wild-type virus. Similar point mutation has been carried out in a plasmid construct encoding the M protein of other flaviviruses, in particular of Zika virus to prepare mutated ZIKV particles. Interestingly, when mosquito cells were infected, the inventors did not observe any difference between the wild-type and the mutant viruses infectious cycles. In order to elucidate at which step of the viral life cycle the mutant viral particles are impaired in their production in mammalian cells, the inventors examined the entry, replication and assembly of WNV in terms of infectious particles production and RNA transcription. The inventors showed that the mutations in the M protein strongly impacted the assembly of genuine viral particles in mammalian cells. Moreover, the mutant virus was severely attenuated in vivo in a mouse model of viral encephalitis, when compared to the wild-type virus.

The inventors thus identified two amino acid residues at position 36 and 43 in the endogenous M protein of wild-type WNV (SEQ ID NO: 2 or SEQ ID NO: 21) that play a major role in the assembly of WNV particles in mammalian cells. More particularly, the inventors found that the replacement of the amino acid which is at position 36 in the ectodomain of the M protein (ectoM) of WNV by an amino acid other than isoleucine (I), more particularly by the amino acid phenylalanine (F), and of the amino acid which is at position 43 in the transmembrane domain 1 of the M protein (TMD1) of WNV by an amino acid other than alanine (A), more particularly by the amino acid glycine (G), leads to attenuation.

Interestingly, these mutations did not impact particle assembly in mosquito cells suggesting different mechanisms/cellular partner(s) for viral particle assembly between mammals and mosquitoes. These results indicated that the M protein of WNV, in particular the ectoM and TMD1 of WNV, contained viral determinants for viral attenuation.

Surprisingly, the amino acid at position 36 in the ectoM of the M protein of WNV alone is key to viral attenuation, but its substitution by an F residue is not stable and quickly reverts to wild type (I residue), both in vitro and in vivo. The amino acid at position 43 in the TMD1 of the M protein of WNV alone does not impact the virus life cycle, but is mandatory to stabilize the amino acid at position 36.

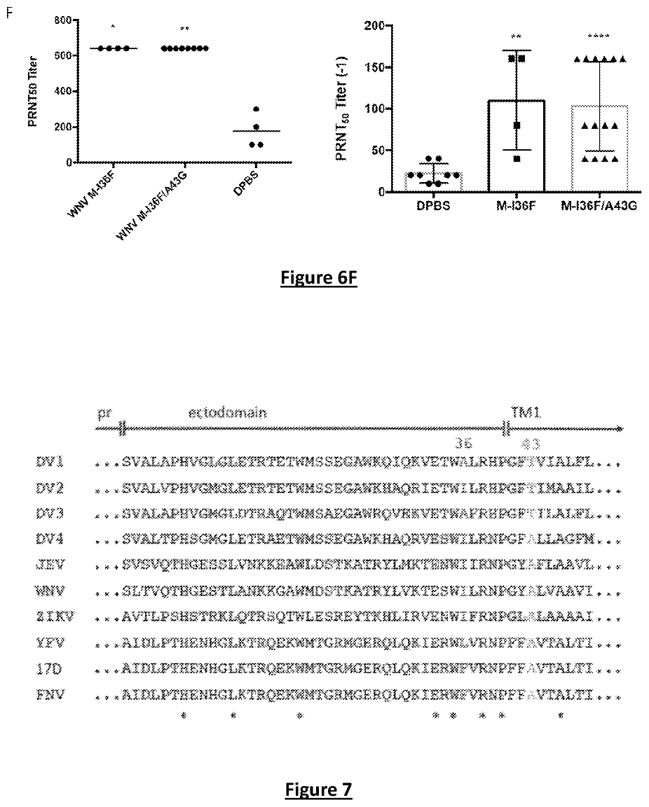

As shown in FIG. 7, the amino acids and positions 36 and 43 of the M protein of WNV are conserved in Dengue virus 4 (DV4), Japanese Encephalitis Virus (JEV), and Zika virus (ZIKV).

The application accordingly relates to a live and attenuated flavivirus, such as a WNV, Dengue virus 4 (DV4), Japanese Encephalitis Virus (JEV), or Zika virus (ZIKV), which is obtainable by mutation of the endogenous M protein of a flavivirus, wherein said mutation comprises or consists of the replacement of the amino acids at positions 36 and 43 in the sequence of said endogenous M protein that correspond to positions 36 and 43 of SEQ ID NO: 2 or SEQ ID NO: 21 in the case of the wild-type WNV (i.e., at positions 251 and 258 in the sequence of the endogenous polyprotein sequence of said wild-type and/or infectious and/or virulent WNV), or at positions corresponding to positions 36 and 43 within the sequence of the endogenous protein M in the case of another wild type flavivirus (i.e. at positions 240 and 247 in the sequence of the endogenous polyprotein sequence of the wild type DV4, or at positions 255 and 262 in the sequence of the endogenous polyprotein sequence of the wild type JEV, or at positions 251 and 258 in the sequence of the endogenous polyprotein sequence of the wild type ZIKV). In particular, the amino acid at the position corresponding to amino acid position 36 of SEQ ID NO: 2 is replaced by an amino acid other than isoleucine (I) and the amino acid at the position corresponding to amino acid position 43 of SEQ ID NO: 2 is replaced by an amino acid other than alanine (A). In a preferred embodiment the amino acid at the position corresponding to amino acid position 36 of SEQ ID NO: 2 is replaced by an amino acid selected from the group consisting of phenylalanine (F), tryptophan (W), and tyrosine (Y), and the amino acid at the position corresponding to amino acid position 43 of SEQ ID NO: 2 is replaced by glycine (G). In a more preferred embodiment the amino acid at the position corresponding to amino acid position 36 of SEQ ID NO: 2 is replaced by phenylalanine (F) and the amino acid at the position corresponding to amino acid position 43 of SEQ ID NO: 2 is replaced by glycine (G).

In a preferred embodiment, the live and attenuated flavivirus is a live and attenuated WNV. The application accordingly relates to a live and attenuated WNV, which is obtainable by mutation of the endogenous M protein of a wild-type WNV, wherein said mutation comprises or consists of the replacement of the amino acids at positions 36 and 43 in the sequence of said endogenous M protein (i.e., at positions 251 and 258 in the sequence of the endogenous polyprotein sequence of said wild-type WNV). In particular, the amino acid at position 36 of SEQ ID NO: 2 (or SEQ ID NO: 21) is replaced by an amino acid other than isoleucine (I) and the amino acid at position 43 of SEQ ID NO: 2 (or SEQ ID NO: 21) is replaced by an amino acid other than alanine (A). In a preferred embodiment the amino acid at position 36 of SEQ ID NO: 2 (or SEQ ID NO: 21) is replaced by an amino acid selected from the group consisting of phenylalanine (F), tryptophan (W), and tyrosine (Y), and the amino acid at position 43 of SEQ ID NO: 2 (or SEQ ID NO: 21) is replaced by glycine (G). In a more preferred embodiment the amino acid at the position corresponding to amino acid position 36 of SEQ ID NO: 2 (or SEQ ID NO: 21) is replaced by phenylalanine (F) and the amino acid at the position corresponding to amino acid position 43 of SEQ ID NO: 2 (or SEQ ID NO: 21) is replaced by glycine (G).

In another embodiment, the live and attenuated flavivirus is a live and attenuated Dengue Virus 4 (DV4). The application accordingly relates to a live and attenuated DV4, which is obtainable by mutation of the endogenous M protein of a wild-type DV4, wherein said mutation comprises or consists of the replacement of the amino acids at positions 36 and 43 in the sequence of said endogenous M protein. In particular, the amino acid at position 36 of SEQ ID NO: 19 is replaced by an amino acid other than isoleucine (I) and the amino acid at position 43 of SEQ ID NO: 19 is replaced by an amino acid other than alanine (A). In a preferred embodiment, the amino acid at position 36 of SEQ ID NO: 19 is replaced by an amino acid selected from the group consisting of phenylalanine (F), tryptophan (W), and tyrosine (Y), and the amino acid at position 43 of SEQ ID NO: 19 is replaced by glycine (G). In a more preferred embodiment, the amino acid at the position corresponding to amino acid position 36 of SEQ ID NO: 19 is replaced by phenylalanine (F) and the amino acid at the position corresponding to amino acid position 43 of SEQ ID NO: 19 is replaced by glycine (G).

In another embodiment, the live and attenuated flavivirus is a live and attenuated Japanese Encephalitis Virus (JEV). The application accordingly relates to a live and attenuated JEV, which is obtainable by mutation of the endogenous M protein of a wild-type JEV, wherein said mutation comprises or consists of the replacement of the amino acids at positions 36 and 43 in the sequence of said endogenous M protein. In particular, the amino acid at position 36 of SEQ ID NO: 20 is replaced by an amino acid other than isoleucine (I) and the amino acid at position 43 of SEQ ID NO: 20 is replaced by an amino acid other than alanine (A). In a preferred embodiment, the amino acid at position 36 of SEQ ID NO: 20 is replaced by an amino acid selected from the group consisting of phenylalanine (F), tryptophan (W), and tyrosine (Y), and the amino acid at position 43 of SEQ ID NO: 20 is replaced by glycine (G). In a more preferred embodiment, the amino acid at the position corresponding to amino acid position 36 of SEQ ID NO: 20 is replaced by phenylalanine (F) and the amino acid at the position corresponding to amino acid position 43 of SEQ ID NO: 20 is replaced by glycine (G).

In another embodiment, the live and attenuated flavivirus is a live and attenuated Zika Virus (ZIKV). The application accordingly relates to a live and attenuated ZIKV, which is obtainable by mutation of the endogenous M protein of a wild-type ZIKV, wherein said mutation comprises or consists of the replacement of the amino acids at positions 36 and 43 in the sequence of said endogenous M protein. In particular, the amino acid at position 36 of SEQ ID NO: 22 or SEQ ID NO: 84 is replaced by an amino acid other than isoleucine (I) and the amino acid at position 43 of SEQ ID NO: 22 or SEQ ID NO: 84 is replaced by an amino acid other than alanine (A). In a preferred embodiment, the amino acid at position 36 of SEQ ID NO: 22 or SEQ ID NO: 84 is replaced by an amino acid selected from the group consisting of phenylalanine (F), tryptophan (W), and tyrosine (Y), and the amino acid at position 43 of SEQ ID NO: 22 or SEQ ID NO: 84 is replaced by glycine (G). In a more preferred embodiment, the amino acid at the position corresponding to amino acid position 36 of SEQ ID NO: 22 or SEQ ID NO: 84 is replaced by phenylalanine (F) and the amino acid at the position corresponding to amino acid position 43 of SEQ ID NO: 22 or SEQ ID NO: 84 is replaced by glycine (G).

In a more preferred embodiment, the live and attenuated ZIKV, comprises a genome encoding a mutated ZIKV M protein, wherein the mutated ZIKV M protein has an amino acid sequence that comprises or consists of SEQ ID NO: 83.

Advantageously, said mutated M protein replaces an endogenous M protein, more particularly the endogenous M protein of a wild-type ZIKV, more particularly the endogenous M protein, the sequence of which is SEQ ID NO: 22 or SEQ ID NO: 84.

In other words, the live and attenuated ZIKV does advantageously not comprise (nor codes for) the endogenous M protein of a wild type ZIKV, more particularly the M protein of SEQ ID NO: 22 or SEQ ID NO: 84.

A particular nucleotide sequence of the polynucleotide encoding said mutated ZIKV M protein as well as a particular amino acid sequence of said mutated ZIKV M protein are the sequences disclosed as SEQ ID NO: 89 and SEQ ID NO: 83 respectively.

SEQ ID NO: 83 (mutated ZIKV M protein of the application):
AVTLPSHSTRKLQTRSQTWLESREYTKHLIKVENWXFRNPGFZLVAVAIAWLLGSST
SQKVIYLVMILLIAPAYS
wherein X = F (phenylalanine), or W (tryptophan) or Y (tyrosine) and Z = G (glycine).
SEQ ID NO: 89 (cDNA sequence coding for a mutated ZIKV MR766 strain M protein
of the application):
CCGTGACGCTCCCTTCTCACTCTACAAGGAAGTTGCAAACGCGGTCGCAGACCTGGTTAGAATCAAGAG
AATACACGAAGCACTTGATCAAGGTTGAAAACTGGnnn₁TTCAGGAACCCCGGGTTTnnn₂CTAGTGGCC
GTTGCCATTGCCTGGCTTTTGGGAAGCTCGACGAGCCAAAAAGTCATATACTTGGTCATGATACTGCT
GATTGCCCCGGCATACAG
wherein nnn₁ = a codon coding either for phenylalanine (TTT or TTC), or tryptophan
(TGG), or tyrosine (TAA, TAG or TGA) and nnn₂ = a codon coding for glycine (GGA,
GGT, GGC orGGG).

As defined herein, the expressions “live and attenuated WNV” and “live attenuated WNV” designate a WNV that is able to replicate in cultured neuroblastoma-derived cells (SK-N-SH), accumulates in the blood of BALB/c mice following inoculation by viral particles, induces production of WNV neutralizing antibodies (seroneutralization) in BALB/c mice following inoculation by viral particles, and induces a protective immune response in BALB/c mice following inoculation with an effective amount of viral particles such that at least 50% of inoculated mice survive a viral challenge with 1000 FFU of WNV WT at 28 days pi.

As defined herein, the expressions “live and attenuated flavivirus” and “live attenuated flavivirus” designate a flavivirus that has attributes equivalent to a live and attenuated WNV.

All the definitions directed to a live and attenuated WNV and mentioned in this application also apply to another flavivirus, such as Dengue virus 4 (DV4), Japanese Encephalitis Virus (JEV), Zika virus (ZIKV) or Usutu virus (USUV).

The wild-type WNV to be mutated for attenuation can e.g., be a WNV Israel strain from 1998 (WNV IS98-ST1) (GENBANK® accession number AF481864). The nucleotide sequence of the polynucleotide encoding the endogenous M protein of WNV IS98-ST1 is presented below as SEQ ID NO: 1. The amino acid sequence of the endogenous M protein of WNV IS98-ST1 is presented below as SEQ ID NO: 2.

SEQ ID NO: 1 (cDNA sequence of the endogenous protein M of WNV IS98-ST1):
TCACTGACAGTGCAGACACACGGAGAAAGCACTCTAGCGAACAAGAAGGGGGCTTGGATGGA
CAGCACCAAGGCCACAAGGTATTTGGTAAAAACAGAATCATGGATCTTGAGGAACCCTGGAT
ATGCCCTGGTGGCAGCCGTCATTGGTTGGATGCTTGGGAGCAACACCATGCAGAGAGTTGTGT
TTGTCGTGCTATTGCTTTTGGTGGCCCCAGCTTACAGC
SEQ ID NO: 2 (endogenous protein M of WNV IS98-ST1):
SLTVQTHGESTLANKKGAWMDSTKATRYLVKTESWILRNPGYALVAAVIGWMLGSNTMQRVVF
VVLLLLVAPAYS

The live and attenuated WNV of the application can e.g., be a WNV of lineage 1.

In an aspect, this application provides a live and attenuated flavivirus comprising a genome encoding a mutated M protein having an amino acid sequence that is at least 93%, or at least 94%, or at least 95%, or at least 96%, or at least 97% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.

The expression “the position corresponding to amino acid position 36 [or 43] of SEQ ID NO: 2” means that the designated sequence of SEQ ID NO: 2 is provided as a reference for the identification of the position of the mutated amino acid residues in the sequence of the M protein of the relevant flavivirus. It is apparent from the sequences illustrated for various flaviviruses in FIG. 7 that mutated positions 36 and 43 in the WNV are also the positions targeted for the mutations in the sequence of the other viruses, i.e. for the replacement of the determined amino acids. Accordingly, when the flavivirus is a specific flavivirus as disclosed in the present application, such as WNV, ZIKV, DV4, JEV, or USUV the sequence of the M protein that is mutated is the sequence encoding the M protein of this particular virus wherein the mutated positions are determined by comparison (such as by alignment provided in FIG. 7) to the respective position of the mutations indicated in SEQ ID NO: 2. These mutated amino acids are generally also located at position 36 and position 43 in the sequence of the M protein in the particular virus.

Also provided is a live and attenuated flavivirus comprising a genome encoding a mutated M protein having an amino acid sequence that is at least 97% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.

In a preferred embodiment, the live and attenuated flavivirus comprises a genome encoding a mutated M protein having an amino acid sequence that is at least 93%, or at least 94%, or at least 95%, or at least 96%, or at least 97% identical to the sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

In a further preferred embodiment, the live and attenuated flavivirus comprises a genome encoding a mutated M protein having an amino acid sequence that consists of the amino acid sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

In a more preferred embodiment, the live and attenuated flavivirus comprises a genome encoding a mutated M protein, wherein the mutated M protein comprises an amino acid of sequence of from 8 to 49 amino acids, comprises an amino acid of sequence of from 8 to 15 amino acids, or comprises an amino acid sequence of from 8 to 25 amino acids of an amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84; encompassing a peptide:
wherein the amino acid at the position corresponding to amino acid position 36 of the amino acid sequence selected from the group consisting of respectively SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by an amino acid selected from the group consisting of phenylalanine, tryptophan and tyrosine; and wherein the amino acid at the position corresponding to amino acid position 43 of the amino acid sequence selected from the group consisting of respectively SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine; or,
wherein the amino acid at the position corresponding to amino acid position 36 of the amino acid sequence selected from the group consisting of respectively SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by phenylalanine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine.

(endogenous M protein of Dengue Virus 4 (DV4))
SEQ ID NO: 19
SVALTPHSGMGLETRAETWMSSEGAWKHAQRVESWILRNPGFALLAGFMAYMIGQTGIQRTV
FFVLMMLVAPSYG
(endogenous M protein of Japanese Encephalitis Virus (JEV))
SEQ ID NO: 20
SVSVQTHGESSLVNKKEAWLDSTKATRYLMKTENWIIRNPGYAFLAAVLGWMLGSNNGQRVV
FTILLLLVAPAYS
(truncated endogenous M protein of West Nile Virus (WNV))
SEQ ID NO: 21
SLTVQTHGESTLANKKGAWMDSTKATRYLVKTESWILRNPGYALVAAVIGWMLGSNTMQRW
FVVLLLLVAPAYS
(endogenous M protein of Zika Virus (ZIKV), PF-13 strain)
SEQ ID NO: 22
AVTLPSHSTRKLQTRSQTWLESREYTKHLIRVENWIFRNPGLALAAAAIAWLLGSSTSQKVI
YLVMILLIAPAYS
(endogenous M protein of Zika Virus (ZIKV), MR766 strain)
SEQ ID NO: 84
AVTLPSHSTRKLQTRSQTWLESREYTKHLIKVENWIFRNPGFALVAVAIAWLLGSST
SQKVIYLVMILLIAPAYS

The nucleotide sequence of the polynucleotide encoding the endogenous M protein of Zika Virus (ZIKV), MR766 strain is presented below as SEQ ID NO: 88.

CCGTGACGCTCCCTTCTCACTCTACAAGGAAGTTGCAAACGCGGTCGCA
GACCTGGTTAGAATCAAGAGAATACACGAAGCACTTGATCAAGGTTGAA
AACTGGATATTCAGGAACCCCGGGTTTGCGCTAGTGGCCGTTGCCATTG
CCTGGCTTTTGGGAAGCTCGACGAGCCAAAAAGTCATATACTTGGTCAT
GATACTGCTGATTGCCCCGGCATACAG
wherein the nucleotide ATA in bold = codon coding for the amino acid at position 36, and the nucleotide GCG in bold = codon coding for the amino acid at position 43.

Advantageously, the mutated M protein replaces an endogenous (wild type) M protein of the flavivirus. In other words, the live and attenuated flavivirus, such as the live and attenuated WNV of the present application, does advantageously not comprise (nor codes for) the endogenous (wild type) M protein of the flavivirus. Accordingly, in a preferred embodiment of the above described flaviviruses comprising in their genome a sequence encoding the mutated M protein as disclosed, the live and attenuated flavivirus is a Dengue Virus 4 (DV4) when the sequence encoding the M protein is SEQ ID NO: 19, Japanese Encephalitis Virus (JEV) when the sequence encoding the M protein is SEQ ID NO: 20, a West Nile Virus (WNV) when the sequence encoding the M protein is SEQ ID NO: 21, a Zika Virus (ZIKV) when the sequence encoding the M protein is SEQ ID NO: 22 or SEQ ID NO: 84 and a Usutu Virus (USUV) when the sequence encoding the M protein is SEQ ID NO: 86.

In some embodiments, the live and attenuated flavivirus shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].

In some embodiments, the live and attenuated flavivirus induces flavivirus neutralizing antibodies following administration to a mammalian host.

In another aspect, live and attenuated WNVs are provided.

In an embodiment, the live and attenuated West Nile Virus (WNV), comprises a genome encoding a mutated WNV M protein having an amino acid sequence that is at least 93%, at least 94%, at least 95%, at least 96%, or at least 97% identical to the sequence of SEQ ID NO: 2; wherein the amino acid at position 36 of SEQ ID NO: 2 is replaced by an amino acid selected from the group consisting of phenylalanine, tryptophan and tyrosine; and wherein the amino acid at position 43 of SEQ ID NO: 2 is replaced by glycine.

In a preferred embodiment, the live and attenuated West Nile Virus (WNV), comprises a genome encoding a mutated WNV M protein having an amino acid sequence that is at least 93%, at least 94%, at least 95%, at least 96%, or at least 97% identical to the sequence of SEQ ID NO: 2; wherein the amino acid at position 36 of SEQ ID NO: 2 is replaced by phenylalanine; and wherein the amino acid at position 43 of SEQ ID NO: 2 is replaced by glycine.

In a more preferred embodiment, the live and attenuated West Nile Virus (WNV), comprises a genome encoding a mutated WNV M protein, wherein the mutated WNV M protein has an amino acid sequence that comprises or consists of SEQ ID NO: 4.

Advantageously, said mutated M protein replaces an endogenous M protein, more particularly the endogenous M protein of a wild-type WNV, more particularly the endogenous M protein, the sequence of which is SEQ ID NO: 2.

In other words, the live and attenuated WNV does advantageously not comprise (nor codes for) the endogenous M protein of a wild type WNV, more particularly the M protein of SEQ ID NO: 2.

Otherwise stated the invention thus described relates to a live attenuated flavivirus the genome of which is mutated in the polynucleotide encoding the M protein and the mutation in said polynucleotide is as disclosed in the present disclosure.

A particular nucleotide sequence of the polynucleotide encoding said mutated WNV M protein as well as a particular amino acid sequence of said mutated WNV M protein are the sequences disclosed as SEQ ID NO: 3 and SEQ ID NO: 4 respectively.

In some embodiments, the live and attenuated WNV shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™]. In some embodiments, the live and attenuated WNV induces WNV neutralizing antibodies following administration to a mammalian host.

SEQ ID NO: 3 (cDNA sequence coding for a mutated WNV M protein of the
application):
TCACTGACAGTGCAGACACACGGAGAAAGCACTCTAGCGAACAAGAAGGGGGCTTGGATGGA
CAGCACCAAGGCCACAAGGTATTTGGTAAAAACAGAATCATGGnnn₁TTGAGGAACCCTGGAT
ATnnn₂CTGGTGGCAGCCGTCATTGGTTGGATGCTTGGGAGCAACACCATGCAGAGAGTTGTGT
TTGTCGTGCTATTGCTTTTGGTGGCCCCAGCTTACAGC
wherein nnn₁ = a codon coding either for phenylalanine (TTT or TTC), or tryptophan
(TGG), or tyrosine (TAA, TAG or TGA) and nnn₂ = a codon coding for glycine (GGA,
GGT, GGC or GGG).
SEQ ID NO: 4 (mutated WNV M protein of the application):
SLTVQTHGESTLANKKGAWMDSTKATRYLVKTESWXLRNPGYZLVAAVIGWMLGSNTMQRVV
FVVLLLLVAPAYS
wherein X = F (phenylalanine), or W (tryptophan) or Y (tyrosine) and Z = G (glycine).

In other words, the live and attenuated WNV of the application can e.g., be a WNV, which comprises or codes for a (mutated WNV) M protein, wherein said (mutated WNV) protein M comprises or consists of the protein of SEQ ID NO: 4.

In a particular embodiment, the live and attenuated WNV of the application comprises the RNA version of the (cDNA) nucleotide sequence of SEQ ID NO: 3 (the sequence of SEQ ID NO: 3 codes for a mutated ectoM and TMD1 of the application; cf. below).

In a particular embodiment, the live and attenuated WNV of the application comprises the RNA version of the (cDNA) nucleotide sequence insert carried by the plasmids STBL3/pUC57 IS98 5′-NS1 (M-I36F/A43G) which has been deposited under the terms of the Budapest Treaty at the Collection Nationale de Culture de Microorganismes (CNCM) under deposit number I-5412, on Mar. 25, 2019.

The plasmid STBL3/pCR2.1 Rep IS98-Gluc has been deposited under the terms of the Budapest Treaty at the Collection Nationale de Culture de Microorganismes (CNCM) under deposit number 1-5477, on Jan. 17, 2020. This plasmid construct contains a fragment of the non-secreted form of Gaussia luciferase (Gluc) reporter gene, foot and mouth disease virus (FMDV)-2A peptide, all non-structural proteins, the first 31 aa of the C protein, the last 25 aa of E protein and the two viral UTRs of the WNV IS98 strain and Hepatitis Delta Virus (HDV) ribozyme.

The address of CNCM is: Collection Nationale de Culture de Microorganismes, Institut Pasteur, 28 rue du Dr Roux, 75724 Paris CEDEX 15, France.

Plasmid stbl3/pUC57 IS98 5′-NS1 (M-I36F/A43G) deposited at the CNCM under deposit number I-5412 was obtained from the plasmid IS98-5′UTR-NS1/pUC57 that contains a SP6 promotor, the 5′UTR end, the structural proteins (C, prM and E) and the N-terminus of NS1 of WNV IS98 strain until the BspEI restriction site, mutated by replacement of the codons, which in the protein M code for the amino acid at positions 36 (i.e., isoleucine) and 43 (i.e alanine), by codons coding respectively for the amino acid phenylalanine (I36F mutation) and glycine (A43G mutation).

The expression “RNA version of a (cDNA) nucleotide sequence” means the (RNA) sequence, which results from the replacement of each nucleotide T of said cDNA nucleotide sequence by the nucleotide U.

Advantageously, the live and attenuated flavivirus of the application, including for example the live and attenuated WNV of the application, shows a default or defect in the assembly of the viral particles (e.g., a reduced production rate of (correctly) assembled viral particles).

Advantageously, the live and attenuated flavivirus of the application, including for example the live and attenuated WNV of the application shows said default or defect in a mammalian cell, but not in a mosquito cell.

By comparison, an infectious WNV (such as the WNV IS98-ST1) does not show this defect in a mosquito cell and does neither show it in a mammalian cell.

In other words, the live and attenuated flavivirus of the application, including for example the live and attenuated WNV of the application shows a default or defect in the assembly of the viral particles (e.g., a reduced production rate of (correctly) assembled viral particles), compared to an infectious WNV (such as the WNV 1S98-ST1; complete genome of WNV IS98-ST1=GENBANK® accession number AF481864; polyprotein of WNV IS98-ST1=GENBANK® accession number AF481864).

Said mammalian cell can e.g., be a rodent cell (such as a mouse cell), a monkey cell, a Cercopithecinae cell, a Cercopithecus aethiops cell (e.g., a cell of the Vero cell line [ATCC® CCL-81™]) or a human cell (e.g., a cell of the HEK293T cell line [ATCC® CRL-3216™] or of the SK-N-SH cell line [ATCC® HTB-11™]). More particularly, said mammalian cell can e.g., be a human cell (e.g., a cell of the HEK293T cell line [ATCC® CRL-3216™] and/or of the SK-N-SH cell line [ATCC® HTB-11 TM]).

Said mosquito cell can e.g., be an Aedes cell, an Aedes albopictus cell or a cell of the C6/36 cell line [ATCC® CRL-1660™].

In an embodiment, the live and attenuated flavivirus of the application, including for example the live and attenuated WNV of the application, can be produced either in mammalian cells (e.g., a cell of the HEK293T cell line [ATCC® CRL-3216™] and/or of the VERO cell line [ATCC® CCL-81™]), or in a mosquito cell (e.g., be an Aedes cell, an Aedes albopictus, or a cell of the C6/36 cell line [ATCC® CRL-1660™]).

In an embodiment, the live and attenuated flavivirus of the application, including for example the live and attenuated WNV of the application, shows said default or defect in a cell of the HEK293T cell line [ATCC® CRL-3216™] and/or of the SK-N-SH cell line [ATCC® HTB-11™]), but not in a cell of the 06/36 cell line [ATCC® CRL-1660™].

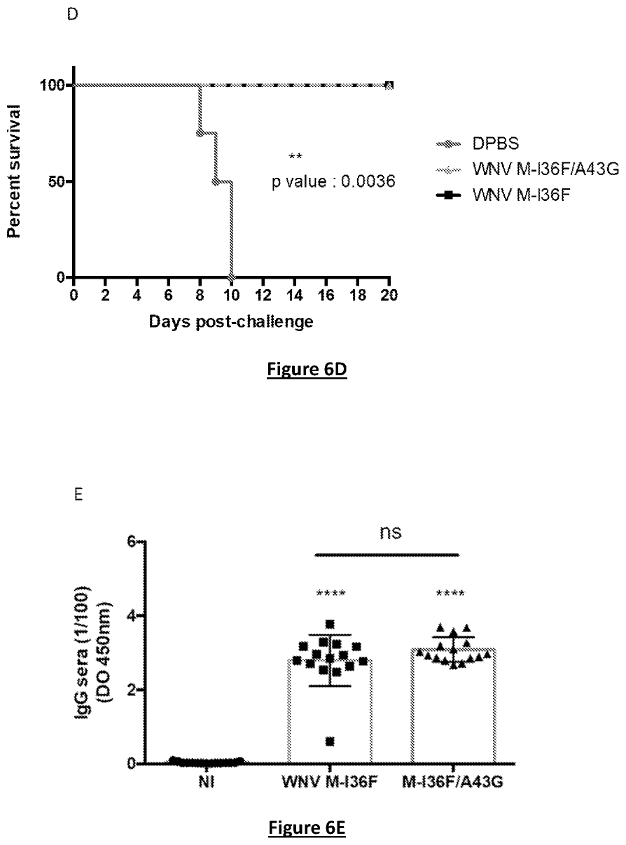

As demonstrated, for example, in the non-limiting embodiment shown in Example 1 below, and FIG. 6A, a 100% survival rate of mice that have received the live and attenuated WNV of the application is achieved. The Example further demonstrates that the mutations at positions 36 and 43 of the protein M of WNV are sufficient to achieve said 100% survival rate. Without wishing to be bound by theory, it is believed that comparable results are achieved with other flaviviruses such as those listed in the present application.

In an embodiment, the live and attenuated WNV of the application induces WNV neutralizing antibodies, more particularly WNV sero-neutralization, more particularly in a mammalian host (such as a rodent, a monkey or a human). This is demonstrated, for example, in the non-limiting embodiment shown in Example 1, and FIG. 6E.

This application also provides the mutated flavivirus M protein of the live and attenuated flavivirus of the application, including for example to the mutated WNV M protein of the live and attenuated WNV, Dengue virus 4 (DV4), Japanese Encephalitis Virus (JEV), Zika virus (ZIKV) or Usutu virus (USUV) of the application.

Accordingly, the application also provides a mutated M protein of a flavivirus, such as a WNV, Dengue virus 4 (DV4), Japanese Encephalitis Virus (JEV), or Zika virus (ZIKV), which is obtainable by mutation of the endogenous M protein of a flavivirus, wherein said mutation comprises or consists of the replacement of the amino acids at positions 36 and 43 in the sequence of said endogenous M protein that correspond to positions 36 and 43 of SEQ ID NO: 2 (i.e., at positions 251 and 258 in the sequence of the endogenous polyprotein sequence of said wild-type WNV), or at positions corresponding to positions 36 and 43 within the sequence of the endogenous protein M in the case of another wild type flavivirus (i.e. at positions 240 and 247 in the sequence of the endogenous polyprotein sequence of the wild type DV4, or at positions 255 and 262 in the sequence of the endogenous polyprotein sequence of the wild type JEV, or at positions 251 and 258 in the sequence of the endogenous polyprotein sequence of the wild type ZIKV). In particular, the amino acid at the position corresponding to amino acid position 36 of SEQ ID NO: 2 is replaced by an amino acid other than isoleucine (I) and the amino acid at the position corresponding to amino acid position 43 of SEQ ID NO: 2 is replaced by an amino acid other than alanine (A). In a preferred embodiment, the amino acid at the position corresponding to amino acid position 36 of SEQ ID NO: 2 is replaced by an amino acid selected from the group consisting of phenylalanine (F), tryptophan (W), and tyrosine (Y), and the amino acid at the position corresponding to amino acid position 43 of SEQ ID NO: 2 is replaced by glycine (G). In a more preferred embodiment, the amino acid at the position corresponding to amino acid position 36 of SEQ ID NO: 2 is replaced by phenylalanine (F) and the amino acid at the position corresponding to amino acid position 43 of SEQ ID NO: 2 is replaced by glycine (G).

In a preferred embodiment, the mutated flavivirus M protein is a mutated WNV M protein. The application accordingly relates to a mutated M protein of a WNV, which is obtainable by mutation of the endogenous M protein of a wild-type WNV, wherein said mutation comprises or consists of the replacement of the amino acids at positions 36 and 43 in the sequence of said endogenous M protein (i.e., at positions 251 and 258 in the sequence of the endogenous polyprotein sequence of said wild-type WNV). In particular, the amino acid at position 36 of SEQ ID NO: 2 is replaced by an amino acid other than isoleucine (I) and the amino acid at position 43 of SEQ ID NO: 2 is replaced by an amino acid other than alanine (A). In a preferred embodiment, the amino acid at position 36 of SEQ ID NO: 2 is replaced by an amino acid selected from the group consisting of phenylalanine (F), tryptophan (W), and tyrosine (Y), and the amino acid at position 43 of SEQ ID NO: 2 is replaced by glycine (G). In a more preferred embodiment, the amino acid at the position corresponding to amino acid position 36 of SEQ ID NO: 2 is replaced by phenylalanine (F) and the amino acid at the position corresponding to amino acid position 43 of SEQ ID NO: 2 is replaced by glycine (G).

The wild-type WNV M protein to be mutated for attenuation can e.g., be the M protein from a WNV Israel strain from 1998 (WNV IS98-ST1) (GENBANK® accession number AF481864).

The nucleotide sequence of the polynucleotide encoding the endogenous M protein of WNV IS98-ST1 is presented herein as SEQ ID NO: 1. The amino acid sequence of the endogenous M protein of WNV IS98-ST1 is presented below as SEQ ID NO: 2.

The WNV of the application can e.g., be an M protein from a WNV of lineage 1.

In an aspect, this application provides a mutated M protein having an amino acid sequence that is at least 93%, at least 94%, at least 95%, at least 96%, or at least 97% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.

Also provided is a mutated M protein having an amino acid sequence that is at least 97% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.

In a preferred embodiment, the mutated M protein having an amino acid sequence that is at least 93%, at least 94%, at least 95%, at least 96%, or at least 97% identical to the sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

In a preferred embodiment, the mutated M protein has an amino acid sequence that consists of the amino acid sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

In a more preferred embodiment, the mutated M protein comprises an amino acid of sequence of from 8 to 49 amino acids, comprises an amino acid of sequence of from 8 to 15 amino acids, or comprises an amino acid sequence of from 8 to 25 amino acids of an amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84; encompassing a peptide:
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by an amino acid selected from the group consisting of phenylalanine, tryptophan and tyrosine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine; or,
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by phenylalanine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine.

In an embodiment, the mutated flavivirus M protein is a WNV M protein having an amino acid sequence that is at least 93%, at least 94%, at least 95%, at least 96%, or at least 97% identical to the sequence of SEQ ID NO: 2; wherein the amino acid at position 36 of SEQ ID NO: 2 is replaced by an amino acid selected from the group consisting of phenylalanine, tryptophan and tyrosine; and wherein the amino acid at position 43 of SEQ ID NO: 2 is replaced by glycine.

In a preferred embodiment, the mutated flavivirus M protein is a WNV M protein having an amino acid sequence that is at least 93%, at least 94%, at least 95%, at least 96%, or at least 97% identical to the sequence of SEQ ID NO: 2; wherein the amino acid at position 36 of SEQ ID NO: 2 is replaced by phenylalanine; and wherein the amino acid at position 43 of SEQ ID NO: 2 is replaced by glycine.

In a more preferred embodiment, the mutated WNV M protein has an amino acid sequence that comprises or consists of SEQ ID NO: 4.

The application also relates to a nucleic acid, more particularly a cDNA or RNA nucleic acid, coding for the mutated flavivirus M protein, such as said mutated WNV M protein, of the application, and cells, more particularly recombinant cells transfected or infected by such cDNA, DNA or RNA.

Examples of nucleic acid coding for said (mutated WNV M protein) of the application include the nucleic acid of SEQ ID NO: 3.

Examples of such (recombinant) cells include:
a bacterial cell, such as an E. coli cell,
an insect cell, such as a mosquito cell, an Aedes cell, an Aedes albopictus cell (e.g., a cell of the C6/36 cell line [ATCC® CRL-1660™]),
a mammal cell, especially a rodent cell (such as a mouse cell), a monkey cell, a Cercopithecinae cell, a Cercopithecus aethiops cell (e.g., a cell of the Vero cell line [ATCC® CCL-81™]) or a human cell (e.g., a cell of the HEK293T cell line [ATCC® CRL-3216™] or of the SK-N-SH cell line [ATCC® HTB-11™]), more particularly
a bacterial cell, such as an E. coli cell, or
an insect cell, such as a mosquito cell, an Aedes cell, an Aedes albopictus cell (e.g., a cell of the C6/36 cell line [ATCC® CRL-1660™]),
a mammal cell, especially a rodent cell (such as a mouse cell), a monkey cell, a Cercopithecinae cell, a Cercopithecus aethiops cell (e.g., a cell of the Vero cell line [ATCC® CCL-81™]) or a human cell (e.g., a cell of the HEK293T cell line [ATCC® CRL-3216™] or of the SK-N-SH cell line [ATCC® HTB-11™]).

The cell can be in isolated form. The cell of the application can be contained in a culture medium, more particularly a non-naturally occurring culture medium, e.g., an in vitro cell culture medium, for example a culture medium comprising the Dulbecco's Modified Eagle Medium (DMEM, INVITROGEN) or comprising the Leibovitz's 15 (L15, INVITROGEN) culture medium.

An example of a nucleotide sequence of the polynucleotide encoding said mutated WNV M protein as well as an amino acid sequence of said mutated WNV M protein are the sequences disclosed as SEQ ID NO: 5 and SEQ ID NO: 6 respectively.

SEQ ID NO: 5 (cDNA sequence coding for a mutated WNV M protein of the
application):
TCACTGACAGTGCAGACACACGGAGAAAGCACTCTAGCGAACAAGAAGGGGGCTTGGATGGA
CAGCACCAAGGCCACAAGGTATTTGGTAAAAACAGAATCATGGTTCTTGAGGAACCCTGGAT
ATGGACTGGTGGCAGCCGTCATTGGTTGGATGCTTGGGAGCAACACCATGCAGAGAGTTGTG
TTTGTCGTGCTATTGCTTTTGGTGGCCCCAGCTTACAGC
wherein TTC = a codon coding for phenylalanine and GGA = a codon coding for
glycine.
SEQ ID NO: 6 (mutated WNV M protein of the application):
SLTVQTHGESTLANKKGAWMDSTKATRYLVKTESWXLRNPGYZLVAAVIGWMLGSNTMQRVV
FVVLLLLVAPAYS
wherein X = F (phenylalanine) and Z = G (glycine)

In the live and attenuated WNV of the application, the WNV structural proteins other than protein M, such the WNV protein E and the WNV protein C, can be the WNV structural proteins of an infectious WNV (such as WNV IS98-ST1).

In the live and attenuated WNV of the application, the WNV non-structural proteins, such as the WNV proteins NS1, NS2A, NS2B, NS3, NS4A, NSA4 and NS5, can be the WNV non-structural proteins of an infectious WNV (such as WN IS98-ST1).

Hence, the application also relates to a live and attenuated WNV, which comprises or codes for a mutated WNV polyprotein, wherein the amino acid sequence of said mutated WNV polyprotein comprises the mutated WNV protein M of the application, more particularly the polypeptide or mutated ectoM and TMD1 of the application.

The application thus relates to a live and attenuated WNV, which comprises or codes for a (mutated WNV) polyprotein, wherein the amino acid sequence of said (mutated WNV) polyprotein comprises or consists of the protein of SEQ ID NO: 7 (WN IS98-ST1 polyprotein, wherein protein M is I36F and A43G mutated).

The sequence of SEQ ID NO: 7 is:

MSKKPGGPGKSRAVNMLKRGMPRVLSLIGLKRAMLSLIDGKGPI
RFVLALLAFFRFTAIAPTRAVLDRWRGVNKQTAMKHLLSFKKELGTLTSAINRRSSKQ
KKRGGKTGIAVMIGLIASVGAVTLSNFQGKVMMTVNATDVTDVITIPTAAGKNLCIVR
AMDVGYMCDDTITYECPVLSAGNDPEDIDCWCTKSAVYVRYGRCTKTRHSRRSRRSLT
VQTHGESTLANKKGAWMDSTKATRYLVKTESWFLRNPGYGLVAAVIGWMLGSNTMQRV
VFVVLLLLVAPAYSFNCLGMSNRDFLEGVSGATWVDLVLEGDSCVTIMSKDKPTIDVK
MMNMEAANLAEVRSYCYLATVSDLSTKAACPTMGEAHNDKRADPAFVCRQGVVDRGWG
NGCGLFGKGSIDTCAKFACSTKAIGRTILKENIKYEVAIFVHGPTTVESHGNYSTQVG
ATQAGRFSITPAAPSYTLKLGEYGEVTVDCEPRSGIDTNAYYVMTVGTKTFLVHREWF
MDLNLPWSSAGSTVWRNRETLMEFEEPHATKQSVIALGSQEGALHQALAGAIPVEFSS
NTVKLTSGHLKCRVKMEKLQLKGTTYGVCSKAFKFLGTPADTGHGTVVLELQYTGTDG
PCKVPISSVASLNDLTPVGRLVTVNPFVSVATANAKVLIELEPPFGDSYIVVGRGEQO
INHHWHKSGSSIGKAETTTLKGAQRLAALGDTAWDFGSVGGVFTSVGKAVHQVFGGAE
RSLFGGMSWITQGLLGALLLWMGINARDRSIALTFLAVGGVLLFLSVNVHADTGCAID
ISRQELRCGNGVFIHNDVEAWMDRYKYYPETPQGLAKIIQKAHKEGVCGLRSVSRLEH
QMWEAVKDELNTLLKENGVDLSVVVEKQEGMYKSAPKRLTATTEKLEIGWKAWGKSIL
FAPELANNTFVVDGPETKECPTQNRAWNSLEVEDFGFGLTSTRMFLKVRESNTTECDS
KIIGTAVKNNLAIHSDLSYWIESRLNDTWKLERAVLGEVKSCTWPETHTLWGDGILES
DLIIPVTLAGPRSNHNRRPGYKTQNQGPWDEGRVEIDFDYCPGTTVTLSESCGHRGPA
TRTTTESGKLITDWCCRSCTLPPLRYQTDSGCWYGMEIRPQRHDEKTLVQSQVNAYNA
DMIDPFQLGLLVVFLATQEVLRKRWTAKISMPAILIALLVLVFGGITYTDVLRYVILV
GAAFAESNSGGDVVHLALMATFKIQPVFMVASFLKARWTNQENILLMLAAVFFQMAYH
DARQILLWEIPDVLNSLAVAWMILRAITFTTTSNVVVPLLALLTPRLRCLNLDVYRIL
LLMVGIGSLIREKRSAAAKKKGASLLCLALASTGLFNPMILAAGLIACDPNRKRGWPA
TEVMTAVGLMFAIVGGLAELDIDSMAIPMTIAGLMFAAFVISGKSTDMWIERTADISW
ESDAEITGSSERVDVRLDDGENFQLMNDPGAPWKIWMLRMVCLAISAYTPWAILPSVV
GFWITLQYTKRGGVLWDTPSPKEYKKGDTTTGVYRIMTRGLLGSYQAGAGVMVEGVFH
TLWHTTKGAALMSGEGRLDPYWGSVKEDRLCYGGPWKLQHKWNGQDEVQMIVVEPGKN
VKNVQTKPGVFKTPEGEIGAVTLDFPTGTSGSPIVDKNGDVIGLYGNGVIMPNGSYIS
AIVQGERMDEPIPAGFEPEMLRKKQITVLDLHPGAGKTRRILPQIIKEAINRRLRTAV
LAPTRVVAAEMAEALRGLPIRYQTSAVPREHNGNEIVDVMCHATLTHRLMSPHRVPNY
NLFVMDEAHFTDPASIAARGYISTKVELGEAAAIFMTATPPGTSDPFPESNSPISDLQ
TEIPDRAWNSGYEWITEYTGKTVWFVPSVKMGNEIALCLQRAGKKVVQLNRKSYETEY
PKCKNDDWDFVITTDISEMGANFKASRVIDSRKSVKPTIITEGEGRVILGEPSAVTAA
SAAQRRGRIGRNPSQVGDEYCYGGHTNEDDSNFAHWTEARIMPDNINMPNGLIAQFYQ
PEREKVYTMEGEYRLRGEERKNFLELLRTADLPVWLAYKVAAAGVSYHDRRWCFDGPR
TNTILEDNNEVEVITKLGERKILRPRWIDARVYSDHQALKAFKDFASGKRSQIGLIEV
LGKMPEHFMGKTWEALDTMYVVATAEKGGRAHRMALEELPDALQTIALIALLSVMTMG
VFFLLMQRKGIGKIGLGGAVLGVATFFCWMAEVPGTKIAGMLLLSLLLMIVLIPEPEK
QRSQTDNQLAVFLICVMTLVSAVAANEMGWLDKTKSDISSLFGQRIEVKENFSMGEFL
LDLRPATAWSLYAVTTAVLTPLLKHLITSDYINTSLTSINVQASALFTLARGFPFVDV
GVSALLLAAGCWGQVTLTVTVTAATLLFCHYAYMVPGWQAEAMRSAQRRTAAGIMKNA
VVDGIVATDVPELERTTPIMQKKVGQIMLILVSLAAVVVNPSVKTVREAGILITAAAV
TLWENGASSVWNATTAIGLCHIMRGGWLSCLSITWTLIKNMEKPGLKRGGAKGRTLGE
VWKERLNQMTKEEFTRYRKEAIIEVDRSAAKHARKEGNVTGGHSVSRGTAKLRWLVER
RFLEPVGKVIDLGCGRGGWCYYMATQKRVQEVRGYTKGGPGHEEPQLVQSYGWNIVTM
KSGVDVFYRPSECCDTLLCDIGESSSSAEVEEHRTIRVLEMVEDWLHRGPREFCVKVL
CPYMPKVIEKMELLQRRYGGGLVRNPLSRNSTHEMYWVSRASGNVVHSVNMTSQVLLG
RMEKRTWKGPQYEEDVNLGSGTRAVGKPLLNSDTSKINNRIERLRREYSSTWHHDENH
PYRTWNYHGSYDVKPTGSASSLVNGVVRLLSKPWDTITNVTTMAMTDTTPFGQQRVFK
EKVDTKAPEPPEGAKYVLNETTNWLWAFLAREKRPRMCSREEFIRKVNSNAALGAMFE
EQNQWRSAREAVEDPKFWEMVDEEREAHLRGECHTCIYNMMGKREKKPGEFGKAKGSR
AIWFMWLGARFLEFEALGFLNEDHWLGRKNSGGGVEGLGLQKLGYILREVGTRPGGKI
YADDTAGWDTRITRADLENEAKVLELLDGEHRRLARAIIELTYRHKVVKVMRPAADGR
TVMDVISREDQRGSGQVVTYALNTFTNLAVQLVRMMEGEGVIGPDDVEKLTKGKGPKV
RTWLFENGEERLSRMAVSGDDCVVKPLDDRFATSLHFLNAMSKVRKDIQEWKPSTGWY
DWQQVPFCSNHFTELIMKDGRTLVVPCRGQDELVGRARISPGAGWNVRDTACLAKSYA
QMWLLLYFHRRDLRLMANAICSAVPVNWVPTGRTTWSIHAGGEWMTTEDMLEVWNRVW
IEENEWMEDKTPVEKWSDVPYSGKREDIWCGSLIGTRARATWAENIQVAINQVRAIIG
DEKYVDYMSSLKRYEDTTLVEDTVL

In one embodiment, the flavivirus structural proteins other than protein M, such the flavivvirus protein E and the flavivirus protein C, more particularly the protein E, can be mutated, more particularly by one or several point mutations, so as to increase flavivirus attenuation (while retaining viability). (see ref Goo et al, PLoS Pathogen 2017, 13(2) e1006178 for the E glycoprotein, or Kaiser et al, Future Virol 2017, 12, 283-295 for all virulence determinants, including prM, E, all the NS and the 3′UTR.)

Alternatively, other WNV proteins, including non structural proteins, structural proteins other than protein M, such the WNV protein E, more particularly the WNV protein E, can be mutated, more particularly by one or several point mutations, so as to increase WNV attenuation (while retaining viability).

The application also relates to the viral particles or virions of the live attenuated flavivirus of the present application, such as said live attenuated WNV of the application.

The application also relates to a RNA nucleic acid, which is the RNA genomic nucleic acid of the live and attenuated flavivirus of the application, including for example the live attenuated WNV of the application. More particularly, the application relates to the coding sequence (CDS) of said genomic RNA.

The application also relates to a DNA nucleic acid, more particularly to a cDNA nucleic acid, the sequence of which is the retro-transcript or cDNA sequence of the RNA genomic nucleic acid of the application, e.g., according to the universal genetic code. More particularly, the application relates to the coding sequence (CDS) of said DNA or cDNA nucleic acid.

The application also relates to a cell, more particularly a host and/or recombinant cell. The cell of the application comprises the live and attenuated flavivirus of the application, including for example the live attenuated WNV of the application, or the mutated M protein of the application, or the mutated ectoM and TMD1 of the application, or the RNA nucleic acid of the application, or the DNA or cDNA nucleic acid of the application.

The cell of the application can e.g., be a cell, which has been infected, transfected or transformed by the live and attenuated flavivirus of the application, including for example the live attenuated WNV of the application, or the mutated M protein of the application, or the mutated ectoM and TMD1 of the application, or the RNA nucleic acid of the application, or the DNA or cDNA nucleic acid of the application.

The cell of the application can be infected, transfected or transformed by methods well known to the person skilled in the art, e.g., by chemical transfection (calcium phosphate, lipofectamine), lipid-based techniques (liposome), electroporation, photoporation. Said infection, transfection or transformation can be transient or permanent.

Examples of a cell of the application include:
a bacterial cell, such as an E. coli cell,
an insect cell, such as a mosquito cell, an Aedes cell, an Aedes albopictus cell (e.g., a cell of the C6/36 cell line [ATCC® CRL-1660™]),
a mammal cell, especially a rodent cell (such as a mouse cell), a monkey cell, a Cercopithecinae cell, a Cercopithecus aethiops cell (e.g., a cell of the Vero cell line [ATCC® CCL-81™]) or a human cell (e.g., a cell of the HEK293T cell line [ATCC® CRL-3216™] or of the SK-N-SH cell line [ATCC® HTB-11™]), more particularly
a bacterial cell, such as an E. coli cell, or
an insect cell, such as a mosquito cell, an Aedes cell, an Aedes albopictus cell (e.g., a cell of the C6/36 cell line [ATCC® CRL-1660™]), or of the SK-N-SH cell line [ATCC® HTB-11™]).

The cell of the application can be in isolated form. The cell of the application can be contained in a culture medium, more particularly a non-naturally occurring culture medium, e.g., an in vitro cell culture medium, for example a culture medium comprising the Dulbecco's Modified Eagle Medium (DMEM, INVITROGEN) or comprising the Leibovitz's 15 (L15, INVITROGEN) culture medium.

A clone or cDNA clone of the application does advantageously not comprise (nor codes for) the M protein of a wild type flavivirus, in particular of a wild type WNV (such as the WNV M protein of SEQ ID NO: 2).

Advantageously, the clone or cDNA clone of the application shows a viral particle assembly default or defect in a mammalian cell but not in a mosquito cell, as described above or below illustrated.

Advantageously, the clone or cDNA clone of the application induces the production of flavivirus neutralizing antibodies, such as WNV neutralizing antibodies, more particularly WNV sero-neutralization, more particularly in a mammalian host (such as a rodent, a monkey or a human), as described above or below illustrated.

Advantageously, the clone or cDNA clone of the application is a live clone or cDNA clone that is also attenuated.

The application also relates to a culture medium comprising the cell or nucleic acid clone of the application, more particularly to a culture medium comprising the cDNA clone of the application. Said culture medium can e.g., be a non-naturally occurring culture medium, e.g., an in vitro cell culture medium, for example a culture medium comprising the Dulbecco's Modified Eagle Medium (DMEM, INVITROGEN) or comprising the Leibovitz's 15 (L15, INVITROGEN) culture medium.

The application also relates to a composition, more particularly a pharmaceutical composition, more particularly an immunogenic composition, more particularly a vaccine, comprising the live and attenuated flavivirus of the application, such as the live and attenuated WNV of the application, or the expression vector of the application, or the cell of the application, or the clone or cDNA clone of the application.

The live and attenuated flavivirus of the application, such as the live and attenuated WNV of the application, the cell of the application, or the clone or cDNA clone of the application can be used as active ingredient for immunization, in particular for prophylactic immunization against a flavivirus infection in a mammalian host, such as a WNV infection in a mammalian host, especially in a human or an animal host.

The live and attenuated flavivirus of the application, such as the live and attenuated WNV of the application, the cell of the application, or the clone or cDNA clone of the application can e.g., be used as active ingredient for prophylactic vaccination against a flavivirus such as WNV.

Advantageously, said composition of the application is suitable for administration into a host, in particular in a mammalian host, especially in a human or an animal host.

Said composition of the application may further comprise a pharmaceutically suitable excipient or carrier and/or vehicle, when used for systemic or local administration.

A pharmaceutically suitable excipient or carrier and/or vehicle refers to a non-toxic solid, semisolid or liquid filler, diluent, encapsulating material or formulation auxiliary of any conventional type. A “pharmaceutically acceptable carrier” is non-toxic to recipients at the dosages and concentrations employed and is compatible with other ingredients of the formulation; suitable carriers include, but are not limited to, phosphate buffered saline solutions, distilled water, emulsions such as an oil/water emulsions, various types of wetting agents sterile solutions and the like, dextrose, glycerol, saline, ethanol, and combinations thereof.

Said composition of the application may further comprise an immunogenic adjuvant, such as Freund type adjuvants, generally used in the form of an emulsion with an aqueous phase or can comprise water-insoluble inorganic salts, such as aluminum hydroxide, zinc sulphate, colloidal iron hydroxide, calcium phosphate or calcium chloride.

In embodiments said composition of the application comprises at least one of the live and attenuated flavivirus of the application such as the live and attenuated WNV of the application, the cell of the application, and the clone or cDNA clone of the application, in a dose sufficient to elicit an immune antibody response, more particularly an immune antibody response against at least one flavivirus polypeptide, such as at least one WNV polypeptide, expressed by the live and attenuated flavivirus of the application such as the live and attenuated WNV of the application, the cell of the application, and/or the clone or cDNA clone of the application. In a particular embodiment, said immune antibody response is a protective humoral response. The protective humoral response results mainly in maturated antibodies, having a high affinity for their antigen, such as IgG. In a particular embodiment, the protective humoral response induces the production of neutralizing antibodies.

It is considered that the composition of the application (in particular the live and attenuated flavivirus of the application such as the live and attenuated WNV of the application) has a protective capacity against flavivirus infection (for example WNV infection) when after challenge of immunized host with the flavivirus (such as WNV), it enables the delay and/or the attenuation of the symptoms usually elicited after infection with said flavivirus (such as WNV) against which protection is sought by the administration of the composition of the application, or when especially the flavivirus infection (such as WNV infection) is delayed.

According to a particular embodiment, said composition of the application is formulated for an administration through parenteral route such as subcutaneous (s.c.), intradermal (i.d.), intramuscular (i.m.), intraperitoneal (i.p.) or intravenous (i.v.) injection, more particularly intraperitoneal (i.p.) injection.

According to another particular embodiment, said composition of the application is administered in one or multiple administration dose(s), in particular in a prime-boost administration regime.

The term “prime-boost regimen” generally encompasses a first administration step eliciting an immune response and one or several later administration step(s) boosting the immune reaction. Accordingly, an efficient prime-boost system can be used for iterative administration, enabling successively priming and boosting the immune response in a host, especially after injections in a host in need thereof.

The term “iterative” means that the active principle is administered twice or more to the host. The priming and boosting immunization can be administered to the host at different or identical doses, and injections can be administered at intervals of several weeks, in particular at intervals of four weeks or more.

The quantity to be administered (dosage) depends on the subject to be treated, including the condition of the patient, the state of the individual's immune system, the route of administration and the size of the host. Suitable dosages can be adjusted by the person of average skill in the art.

The application also relates to a method to treat, prevent and/or protect, against a flavivirus infection (such as a WNV infection) in a mammalian host, especially in a human or a non-human animal host, comprising administering said live and attenuated flavivirus of the application (such as the live and attenuated WNV of the application), or said cell of the application, or said clone or cDNA clone of the application or said composition of the application to said mammalian host.

As used herein, the expression “to protect against WNV infection” refers to a method by which a West Nile virus infection is obstructed or delayed, especially when the symptoms accompanying or following the infection are attenuated, delayed or alleviated, and/or when the infecting virus is cleared from the host. In a similar definition, when the flavivirus is a different virus as disclosed in the present application, the protection against this particular other flavivirus is achieved when the symptoms accompanying or following the infection by such flavivirus are attenuated, delayed or alleviated, and/or when the infecting virus is cleared from the host.

The application also relates to a method to produce a live and attenuated flavivirus, in particular WNV, which comprises producing said live and attenuated flavivirus, in particular WNV, of the application, or said cell of the application, or said clone or cDNA clone of the application or said composition of the application.

The application also relates to a method to produce an immunogenic composition or vaccine against a flavivirus infection, such as a WNV infection, which comprises producing said live and attenuated flavivirus of the application such as the live and attenuated WNV of the application, e.g., as a clone or cDNA clone in a culture medium, optionally collecting the viral particles or virions produced by said live and attenuated flavivirus of the application such as the live and attenuated WNV of the application, and formulating said cultured flavivirus (such as WNV) (or said collected viral particles) in a composition suitable for administration to an animal, more particularly to a human. Said culture medium can e.g., be a non-naturally occurring culture medium, e.g., an in vitro cell culture medium, for example a culture medium comprising the Dulbecco's Modified Eagle Medium (DMEM, INVITROGEN) or comprising the Leibovitz's 15 (L15, INVITROGEN) culture medium.

The application also relates to a method of (in vitro) attenuation of wild type flavivirus (such as a WNV), which comprises or consists of mutating the protein M of said wild type flavivirus (such as WNV), wherein said mutation comprises or consists of the replacement of the amino acids which are at position 36 and 43 within the sequence of said protein M, in the case of a WNV, or the positions corresponding to positions 36 and 43 within the sequence of said protein M, in the case of another flavivirus, by the amino acids phenylalanine (F) or tryptophan (W) or tyrosine (Y) at position 36, and by the amino acid glycine (G) at position 43, more particularly by the amino acids phenylalanine and glycine, respectively.

In some embodiments, the attenuated flavivirus (such as WNV or ZIKV) thus produced still is a live virus.

In some embodiments, the (live and) attenuated flavivirus (such as WNV or ZIKV) thus produced shows a viral particle assembly default or defect in a mammalian cell but not in a mosquito cell.

In some embodiments, the (live and) attenuated WNV or ZIKV thus produced induces the production of WNV or ZIKV neutralizing antibodies, more particularly WNV or ZIKV sero-neutralization, more particularly in a mammalian host (such as a rodent, a monkey or a human), as described above or below illustrated.

The present invention relates in particular to the following embodiments:

1. A live and attenuated flavivirus comprising a genome encoding a mutated M protein having an amino acid sequence that is at least 93% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.
2. The live and attenuated flavivirus according to embodiment 1, wherein the mutated flavivirus M protein has an amino acid sequence that is at least 97% identical to the sequence of the wild type M protein of the flavivirus.
3. The live and attenuated flavivirus according to embodiment 1 or embodiment 2, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.
4. The live and attenuated flavivirus according to any one of embodiments 1 to 3, wherein the mutated flavivirus M protein has an amino acid sequence that consists of the amino acid sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.
5. The live and attenuated flavivirus according to any one of embodiments 1 to 4, wherein the mutated M protein comprises an amino acid of sequence of from 8 to 49 amino acids, comprises an amino acid of sequence of from 8 to 15 amino acids, or comprises an amino acid sequence of from 8 to 25 amino acids of an amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84;
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by an amino acid selected from the group consisting of phenylalanine, tryptophan and tyrosine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine; or
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by phenylalanine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine.
   6. The live and attenuated flavivirus according to any one of embodiments 1 to 5, which does not comprise a protein having the amino acid sequence of SEQ ID NO: 2.
   7. The live and attenuated flavivirus according to any one of embodiments 1 to 6, which does not comprise a genome encoding a protein having the amino acid sequence of SEQ ID NO: 2.
   8. The live and attenuated flavivirus according to any one of embodiments 1 to 7, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   9. The live and attenuated flavivirus according to any one of embodiments 1 to 8, which induces flavivirus neutralizing antibodies following administration to a mammalian host.
   10. A RNA nucleic acid, which is the RNA genomic nucleic acid of the live and attenuated flavivirus according to any one of embodiments 1 to 9.
   11. A cDNA nucleic acid, the sequence of which is the retrotranscript of the RNA genomic nucleic acid of embodiment 9.
   12. A recombinant cell, which comprises the live and attenuated flavivirus of any one of embodiments 1-9, and/or the RNA nucleic acid of claim 10, and/or the cDNA nucleic acid of embodiment 11.
   13. A cDNA clone of the live and attenuated flavivirus of any one of embodiments 1-9.
   14. The cDNA clone of embodiment 13, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   15. The cDNA clone according to embodiment 13 or 14, which induces flavivirus neutralizing antibodies following administration to a mammalian host.
   16. An immunogenic composition, which comprises:
the live and attenuated flavivirus of any one of embodiments 1-9 or viral particles thereof, or
the cDNA clone of any one of embodiments 13-15.
   17. The live and attenuated flavivirus of any one of embodiments 1-9, the RNA nucleic acid of embodiment 10, the cDNA nucleic acid of embodiment 11, the recombinant cell of embodiment 12, the cDNA clone of any one of embodiments 13-15, or the immunogenic composition of embodiment 16, wherein the flavivirus is selected from the group consisting of Dengue virus 4 (DV4), Japanese Encephalitis Virus (JEV), Zika virus (ZIKV) and Usutu virus (USUV), preferably is ZIKV.
   18. A live and attenuated West Nile Virus (WNV), comprising a genome encoding a mutated WNV M protein having an amino acid sequence that is at least 93% identical to the sequence of SEQ ID NO: 2; and wherein the amino acid at position 36 of SEQ ID NO: 2 is replaced by an amino acid selected from the group consisting of phenylalanine, tryptophan and tyrosine; and wherein the amino acid at position 43 of SEQ ID NO: 2 is replaced by glycine.
   19. The live and attenuated WNV according to embodiment 18, wherein the mutated WNV M protein has an amino acid sequence that is at least 97% identical to the sequence of SEQ ID NO: 2.
   20. The live and attenuated WNV according to embodiment 18 or 19, wherein the amino acid at position 36 of SEQ ID NO: 2 is replaced by phenylalanine; and wherein the amino acid at position 43 of SEQ ID NO: 2 is replaced by glycine.
   21. The live and attenuated WNV according to any one of embodiments 18 to 20, wherein the mutated WNV M protein has an amino acid sequence that comprises or consists of SEQ ID NO: 4.
   22. The live and attenuated WNV according to any one of embodiments 18 to 21, which does not comprise a protein having the amino acid sequence of SEQ ID NO: 2.
   23. The live and attenuated WNV according to any one of embodiments 18 to 22, which does not comprise a genome encoding a protein having the amino acid sequence of SEQ ID NO: 2.
   24. The live and attenuated WNV according to any one of embodiments 18 to 23, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   25. The live and attenuated WNV according to any one of embodiments 18 to 24, which induces WNV neutralizing antibodies following administration to a mammalian host.
   26. A RNA nucleic acid, which is the RNA genomic nucleic acid of the live and attenuated WNV according to any one of embodiments 18 to 25.
   27. A cDNA nucleic acid, the sequence of which is the retrotranscript of the RNA genomic nucleic acid of embodiment 26.
   28. A recombinant cell, which comprises the live and attenuated WNV of any one of embodiments 18-25, and/or the RNA nucleic acid of embodiment 26, and/or the cDNA nucleic acid of embodiment 27.
   29. A cDNA clone of the live and attenuated WNV of any one of embodiments 18-25.
   30. The cDNA clone of embodiment 29, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   31. The cDNA clone according to embodiment 29 or 30, which induces WNV neutralizing antibodies following administration to a mammalian host.
   32. An immunogenic composition, which comprises:
the live and attenuated WNV of any one of embodiments 18-25 or viral particles thereof, or
the cDNA clone of any one of embodiments 29-31.
   33. A method to treat, prevent and/or protect, against a flavivirus infection in a mammalian host, comprising administering an effective amount of the immunogenic composition according to embodiment 16 to the host.
   34. A method to treat, prevent and/or protect, against a WNV infection in a mammalian host, comprising administering an effective amount of the immunogenic composition according to embodiment 32 to the host, preferably a human host.
   35. The immunogenic composition according to embodiment 16 for the treatment, prevention and/or protection against a flavivirus infection in a mammalian host, preferably a human host.
   36. The immunogenic composition according to embodiment 32 for the treatment, prevention and/or protection against a WNV infection in a mammalian host, preferably a human host.
   37. A live and attenuated Zika Virus (ZIKV), comprising a genome encoding a mutated ZIKV M protein having an amino acid sequence that is at least 93% identical to the sequence of SEQ ID NO: 22 or SEQ ID NO: 84, preferably SEQ ID NO: 84; and wherein the amino acid at position 36 of SEQ ID NO: 22 or SEQ ID NO: 84, preferably SEQ ID NO: 84, is replaced by an amino acid selected from the group consisting of phenylalanine, tryptophan and tyrosine; and wherein the amino acid at position 43 of SEQ ID NO: 22 or SEQ ID NO: 84, preferably SEQ ID NO: 84, is replaced by glycine.
   38. The live and attenuated ZIKV according to embodiment 37, wherein the mutated ZIKV M protein has an amino acid sequence that is at least 97% identical to the sequence of SEQ ID NO: 22 or SEQ ID NO: 84, preferably SEQ ID NO: 84.
   39. The live and attenuated ZIKV according to embodiment 37 or 38, wherein the amino acid at position 36 of SEQ ID NO: 22 or SEQ ID NO: 84, preferably SEQ ID NO: 84, is replaced by phenylalanine; and wherein the amino acid at position 43 of SEQ ID NO: 22 or SEQ ID NO: 84, preferably SEQ ID NO: 84, is replaced by glycine.
   40. The live and attenuated ZIKV according to any one of embodiments 37 to 39, wherein the mutated ZIKV M protein has an amino acid sequence that comprises or consists of SEQ ID NO: 83.
   41. The live and attenuated ZIKV according to any one of embodiments 37 to 40, which does not comprise a protein having the amino acid sequence of SEQ ID NO: 22 or SEQ ID NO: 84, preferably SEQ ID NO: 84.
   42. The live and attenuated ZIKV according to any one of embodiments 37 to 41, which does not comprise a genome encoding a protein having the amino acid sequence of SEQ ID NO: 22 or SEQ ID NO: 84, preferably SEQ ID NO: 84.
   43. The live and attenuated ZIKV according to any one of embodiments 37 to 42, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   44. The live and attenuated ZIKV according to any one of embodiments 37 to 43, which induces ZIKV neutralizing antibodies following administration to a mammalian host.
   45. A RNA nucleic acid, which is the RNA genomic nucleic acid of the live and attenuated ZIKV according to any one of embodiments 37 to 44.
   46. A cDNA nucleic acid, the sequence of which is the retrotranscript of the RNA genomic nucleic acid of embodiment 45.
   47. A recombinant cell, which comprises the live and attenuated ZIKV of any one of embodiments 37-44, and/or the RNA nucleic acid of embodiment 45, and/or the cDNA nucleic acid of embodiment 46.
   48. A cDNA clone of the live and attenuated ZIKV of any one of embodiments 37-44.
   49. The cDNA clone of embodiment 48, which shows a defect in the assembly of the viral particles in a human cell of the HEK293T cell line [ATCC® CRL-3216™] and/or of the SK-N-SH cell line [ATCC® HTB-11™]), but not in a mosquito cell of the C6/36 cell line [ATCC® CRL-1660™].
   50. The cDNA clone according to embodiment 48 or 49, which induces ZIKV neutralizing antibodies following administration to a mammalian host.
   51. An immunogenic composition, which comprises:
the live and attenuated ZIKV of any one of embodiments 37-44 or viral particles thereof, or
the cDNA clone of any one of embodiments 48-50.
   52. A method to treat, prevent and/or protect, against a ZIKV infection in a mammalian host, comprising administering an effective amount of the immunogenic composition according to embodiment 51 to the host, preferably a human host.
   53. The immunogenic composition according to embodiment 51 for the treatment, prevention and/or protection against a ZIKV infection in a mammalian host, preferably a human host.
   54. A live and attenuated flavivirus selected from the group consisting of respectively Dengue virus 4 (DV4), West Nile virus (WNV), Japanese Encephalitis Virus (JEV), Zika virus (ZIKV) and Usutu virus (USUV), preferably is WNV or ZIKV, the genome of which encodes a mutated M protein, and wherein in said mutated M protein:
the amino acid at the position corresponding to amino acid position 36 of the amino acid sequence selected from the group consisting of respectively SEQ ID NO: 19 (DV4), SEQ ID NO: 20 (JEV), SEQ ID NO: 2 (WNV), SEQ ID NO: 21 (WNV), SEQ ID NO: 22 (ZIKV), SEQ ID NO: 84 (ZIKV) and SEQ ID NO: 86 (USUV) is replaced by an amino acid selected from the group consisting of phenylalanine, tryptophan and tyrosine; and wherein the amino acid at the position corresponding to amino acid position 43 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by glycine; or
the amino acid at the position corresponding to amino acid position 36 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by phenylalanine; and wherein the amino acid at the position corresponding to amino acid position 43 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by glycine.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the application.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### EXAMPLES

### Example 1 (WNV)

### Materials and Methods

### Cells

Green monkey epithelial cells (Vero-E6), human neuroblastoma cells SK-N-SH (ATCC® HTB-11™) and human kidney cells HEK293T (ATCC® CRL-3216™) were cultured at 37° C. in Dulbecco's Modified Eagle Medium (DMEM, INVITROGEN) containing 10% of fetal bovine serum (FBS).

Aedes albopictus cells C6/36 [ATCC® CRL-1660™] were cultured at 28° C. in Leibovitz's 15 (L15, INVITROGEN) medium containing 10% of FBS and 1% of penicillin and streptomycin.

### Plasmids

The previously described two-plasmid infectious clone of WNV IS98-ST1 (Alsaleh et al, 2016) was used to produce WNV WT, and mutants. The plasmid IS98-5′UTR-NS1/pUC57 contains a SP6 promotor, the 5′UTR end, the structural proteins (C, prM and E) and the N-terminus of NS1 of WNV until the BspEI restriction site. The replicon plasmid, Rep-IS98-Gluc/pCR2.1, contains a fragment of the non-secreted form of Gaussia luciferase (Gluc) reporter gene, foot and mouth disease virus (FMDV)-2A peptide, all non-structural proteins, the first 31 aa of the C protein, the last 25 aa of E protein, the two viral UTRs and HDV ribozyme (Figure A). Cell transfection of resulting RNA leads to production of WNV virions.

### Site-Directed Mutagenesis

Site-directed mutagenesis was conducted on plasmid IS98-5′UTR-NS1/pUC57 by PCR using PHUSION High Fidelity polymerase (Thermo Scientific) and the following primers to introduce mutations M-I36F and M-A43G respectively: FW (M-I36F): 5′-AAAACAGAATCATGGTTCTTGAGGAACCCTG-3′ (SEQ ID NO: 8), RV (M-I36F): 5′-CCAGGGTTCCTCAAGAACCATGATTCTGTTTT-3′ (SEQ ID NO: 9) and FW (M-A43G): 5′-ACCCTGGATATGGACTGGTGGCAGC-3′ (SEQ ID NO: 10), RV (M-A43G): 5′-GCTGCCACCAGTCCATATCCAGGGT-3′ (SEQ ID NO: 11).

PCR products were digested with DpnI enzyme (NEW ENGLAND BIOLABS) and used to transform competent bacteria STBL3 (Life Technologies). Bacteria were cultured in medium LB containing 100 mM of carbenicillin at 37° C. overnight.

### Virus Production

Both plasmids, IS98-5′UTR-NS1/pUC57 and Rep-IS98-Gluc/pCR2.1 are stable and can be produced from STBL3 Escherichia coli (Life technologies) at 37° C. They were used to reconstitute the full-length viral genome (two-plasmid infectious clone, see above). The plasmid Rep-IS98-Gluc/pCR2.1 was digested with the restriction enzyme MIul (NEB), dephosphorylated with the Antartic Phosphatase (NEB) and finally digested with the restriction enzyme BspEI (NEB). The plasmid was purified by chloroform/ethanol precipitation after each digestion. The plasmid IS98-5′UTR-NS1/pUC57 was first digested with restriction enzyme Sall (NEB), dephosphorylated with the Antartic Phosphatase (NEB) and purified by chloroform/ethanol precipitation. The plasmid was next digested with BspEI and purified. A final amount of 2 to 2.5 μg of the two plasmids were used for ligation at a ratio of 1:1 using high concentration T4 DNA ligase (NEB) overnight at 16° C. After an inactivation step at 65° C. for 10 min, the ligation product was linearized by BamHI, purified and transcribed in vitro using mMessage mMachine SP6 kit (Ambion) according to the manufacturer's instructions. The resulting RNA was precipitated by LiCl and purified according to manufacturer's instructions. The RNA was then quantified and stored at −80° C. in aliquots of 10 μg.

Wild type (WT) and mutant M-I36F, M-A43G and M-I36F/A43G viruses were produced by electroporation of the resulting RNA (see above) in C6/36 cells or Vero cells using GenePulser Xcell™ Electroporation system (BioRad), according to the supplier's instructions. Supernatants were collected 3 days post-electroporation.

The production of a full-length infectious clone was performed as already described (Alsaleh K, et al., 2016, Virology 492:53-65), purified and transcribed in vitro using the mMessage mMachine™ SP6 kit (ThermoFischer Scientific). The resulting RNA was electroporated in C6/36 cells (400 V, 25 μF, 8000) in OPTI-MEM medium (ThermoFischer Scientific). Cell culture supernatants were collected 72 h post-electroporation and used to infect 10⁷ C6/36 cells. Three-days pi, viral supernatants were amplified by infecting 5×10⁷ C6/36 cells during 3 days before collection and utilization as final viral stocks. Full-length viral genomes were sequenced from cDNA obtained by reverse transcription using Superscript II Reverse Transcription kit (Invitrogen) according to manufacturer's instructions. cDNAs were then amplified by PCR using Phusion High Fidelity kit (ThermoFischer Scientific) and primers presented in supplementary material (Table 1).

TABLE 1
Primers used for the amplification and sequencing of the complete wild-type
and mutant viral genomes, and are disclosed as SEQ ID NOs: 39-82.

| Oligo Forward | Forward Sequence | Tm | Oligo reverse | Reverse Sequence | Tm | Size |
|---|---|---|---|---|---|---|
| 11-32F | ctgtgtgagctgacaaacttag | 55 | 780-797R | caagcccccttcttgttc | 58 | 787 |
| | (SEQ ID NO: 39) | | | (SEQ ID NO: 61) | | |
| 501-520F | gacggtaaatgctactgacg | 56 | 1282-1300R | ttcctttgccaaatagtcc | 55 | 800 |
| | (SEQ ID NO: 40) | | | (SEQ ID NO: 62) | | |
| 1000-1018F | ttggaaggagtgtctggag | 56 | 1781-1800R | agtgttgcttgaaaattcca | 56 | 801 |
| | (SEQ ID NO: 41) | | | (SEQ ID NO: 63) | | |
| 1500-1520F | aaagcttggagaatatggaga | 56 | 2278-2295R | atggacagccttcccaac | 58 | 796 |
| | (SEQ ID NO: 42) | | | (SEQ ID NO: 64) | | |
| 2000-2017F | cattgaacgacctaacgc | 55 | 2766-2783R | gtgaggcgtttaggtgct | 56 | 784 |
| | (SEQ ID NO: 43) | | | (SEQ ID NO: 65) | | |
| 2500-2519F | caagagctgagatgtggaag | 56 | 3281-3298R | agtcaatctctacccggc | 55 | 799 |
| | (SEQ ID NO: 44) | | | (SEQ ID NO: 66) | | |
| 3001-3022F | gaatgtgactcgaagatcattg | 57 | 3777-3799R | ccaccataaacactggttgt | 58 | 799 |
| | (SEQ ID NO: 45) | | | atc | | |
| | | | | (SEQ ID NO: 67) | | |
| 3501-3518F | cctcgtgcagtcacaagt | 56 | 4280-4297R | caatgtcaagctctgcca | 57 | 797 |
| | (SEQ ID NO: 46) | | | (SEQ ID NO: 68) | | |
| 4000-4017F | ctagccctgctaacaccc | 56 | 4781-4798R | cctctccgctcatcaaag | 57 | 799 |
| | (SEQ ID NO: 47) | | | (SEQ ID NO: 69) | | |
| 4500-4519F | ttggaagatatggatgctca | 57 | 5277-5294R | acaaccctggttggtgct | 59 | 795 |
| | (SEQ ID NO: 48) | | | (SEQ ID NO: 70) | | |
| 5003-5020F | ccactggaacatcaggct | 57 | 5783-5800R | ttgggtactccgtctcgt | 57 | 798 |
| | (SEQ ID NO: 49) | | | (SEQ ID NO: 71) | | |
| 5500-5520F | gcaagaggttacatttccaca | 58 | 6282-6299R | cacctccggtcgtggtat | 59 | 800 |
| | (SEQ ID NO: 50) | | | (SEQ ID NO: 72) | | |
| 6001-6018F | agaaatccgtcgcaagtt | 56 | 6783-6800R | gagagcagcaacattccg | 58 | 800 |
| | (SEQ ID NO: 51) | | | (SEQ ID NO: 73) | | |
| 6503-6520F | tgcctgagcacttcatgg | 59 | 7283-7300R | aaccgggaaccatgtagg | 58 | 798 |
| | (SEQ ID NO: 52) | | | (SEQ ID NO: 74) | | |
| 7031-7049F | caacagcctggtcactgta | 55 | 7775-7792R | agcgatcgacttcgatga | 58 | 762 |
| | (SEQ ID NO: 53) | | | (SEQ ID NO: 75) | | |
| 7500-7517F | acgagaagccggaatttt | 57 | 8255-8275R | ggagcagctccatcttctct | 59 | 776 |
| | (SEQ ID NO: 54) | | | a | | |
| | | | | (SEQ ID NO: 76) | | |
| 8000-8017F | gtcccggacatgaagagc | 59 | 8663-8681R | catggttttgagaggagcc | 58 | 682 |
| | (SEQIDNO:55) | | | (SEQ ID NO: 77) | | |
| 8482-8499F | ggaaaacccctgctcaac | 58 | 9283-9300R | ccagccagctgtgtcatc | 59 | 819 |
| | (SEQID NO: 56) | | | (SEQ ID NO: 78) | | |
| 9017-9034F | ggggggaatgtcacactt | 58 | 9782-9800R | tcataccatccagttgacg | 55 | 784 |
| | (SEQID NO: 57) | | | (SEQ ID NO: 79) | | |
| 9500-9517F | ttgtcacctacgccctaa | 55 | 10282-10300R | ggttgatagcca cctggat | 57 | 801 |
| | (SEQID NO: 58) | | | (SEQ ID NO: 80) | | |
| 10002-10019F | aagagacctgcggctcat | 58 | 10783-10800R | tttcgccctggttaacac | 57 | 799 |
| | (SEQID NO: 59) | | | (SEQ ID NO: 81) | | |
| 10504-1052IF | aaagtcaggccgggaagt | 60 | 11011-11028R | atcctgtgttctcgcacc | 57 | 525 |
| | (SEQ ID NO: 60) | | | (SEQ ID NO: 82) | | |

### Antibodies

Monoclonal antibody (mAb) 4G2 anti-Flavivirus E protein and HRP-conjugated mAb 4G2 were purchased from RD Biotech (Besançon, France). Polyclonal anti-WNV was isolated from intraperitoneal liquid of mice infected with WNV. Secondary antibody Horseradish peroxidase (HRP)-conjugated goat anti-mouse IgG was purchased from Bio-Rad Laboratories. Secondary gold-conjugated goat-anti-mouse antibody was purchased from Aurion (Wageningen, Netherlands).

### M Protein 3-Fold Structure

M protein 3D structure data were obtained from the PDB (PDB accession number: 5wsn) and edited using PyMOL program.

### Electroporation

5×10⁶ Vero cells or 10⁷ C6/36 cells were electroporated with 10 μg of synthetized RNAs using the following settings respectively: 1 pulse, 400V, 25 uF, 800 ohm, or 2 pulses, 25 ms, 140V. Cells were resuspended in DMEM containing 2% FBS or L15 containing 2% FBS respectively in a T25 flask. Cell supernatants were harvested 3 days post-electroporation and used to re-infect Vero cells or C6/36 cells for 3 days. Collected supernatants were clarified by centrifugation and stored in aliquots at −80° C.

### Infection

10⁵ SK-N-SH cells or Vero cells were seeded in 24-well-culture plaques. 24 hours later, they were infected with 200 μL of medium containing a given number of viral particles, depending on the MOI. One hour after inoculation, inoculum was replaced by medium containing 2% of FBS.

Titration in ffu/mL

Vero-NK cells were seeded at 8×10⁴ cells per well in 24-well plates and incubated at 37° C. for 24 h. Tenfold dilutions of virus in DMEM were added to the cells and incubated for 1 h at 37° C. Unadsorbed virus was removed, then 1 ml of DMEM supplemented with 1.6% carboxymethyl cellulose (CMC), 10 mM HEPES buffer, 72 mM sodium bicarbonate, and 2% FBS was added to each well, followed by incubation at 37° C. for 2 days. The CMC overlay was removed, the cells were washed with PBS and fixed with 4% paraformaldehyde for 15 min, followed by permeabilization with 0.2% Triton X-100 for 5 min. Cells were then washed with PBS and incubated for 1 h at room temperature (RT) with anti-E antibody (4G2), followed by incubation with HRP-conjugated anti-mouse IgG antibody. The foci were revealed using the Vector VIP peroxidase substrate kit (Vector Laboratories) according to the manufacturer's instructions.

### Western Blotting

Protein lysates were prepared by cell lysis in RIPA buffer (Bio Basic) containing protease inhibitors (Roche). Equal amounts of proteins, or supernatants, were loaded on a NuPAGE Novex 4 to 12% Bis-Tris protein gel (Life Technologies) and transferred to a PVDF membrane (Bio-Rad). After blocking the membrane for 2 h at room temperature in PBS-Tween (PBS-T) plus 5% milk, the blot was incubated overnight at 4° C. with either anti-E protein antibody (1/1000, RD Biotech, Besançon, France) or anti-calnexin antibody (1/1000, Enzo Life Sciences). The membrane was then washed in PBS-T and then incubated for 2 h at RT in the presence of HRP-conjugated secondary antibodies. After washes in PBS-T, the membrane was incubated in the Pierce ECL Western blotting substrate (Thermo Scientific) and the protein bands were revealed using MyECL Imager machine (Thermofisher). When necessary, the bands were quantified using MyImage software (Thermofisher).

### Quantitative RT-PCR

Total RNA were extracted from samples using NucleoSpin RNA (Macherey-Nagel) according to manufacturer's instructions. The RNA standard used for quantification of WNV copy numbers was produced as already described (ref Alsaleh et al, 2016). The quantitation of a given target RNA was performed using 2 μl of RNA and the SYBR green PCR Master Mix kit (ThermoFisher Scientific) according to manufacturer's instructions. The real-time PCR system (Thermofisher scientific) was used to measure SYBR green fluorescence with the following program: reverse transcription step at 48° C. (30 min), followed by an initial PCR activation step at 94° C. (10 min), 40 cycles of denaturation at 94° C. (15 s) and annealing at 60° C. (30 s). Results were analyzed using the CFX Manager software (Bio-Rad). Primers 5′-GCGGCAATATTCATGACAGCC-3′ (SEQ ID NO: 12) and 5′-CGGGATCTCAGTCTGTAAGTC-3′ (SEQ ID NO: 13) were used for viral genome quantification. Target gene expression was normalized to the expression of GAPDH mRNA, measured using the 2 following primers: 5′-GGTCGGAGTCAACGGATTTG-3′ (SEQ ID NO: 14) and 5′-ACTCCACGACGTACTCAGCG-3′ (SEQ ID NO: 15).

### Viral Entry Assay

SK-N-SH cells (10⁵) or C6/36 cells (5×10⁵) were seeded in a 24-wells plate and grown overnight at 37° C. or 28° C. respectively. Cells were placed on ice for 30 minutes and washed two-times with cold DPBS. Cells on ice were infected with either WNV WT, M-A43G, M-I36F or M-I36F/A43G at a MOI of 10 diluted in cold DMEM or L15 containing 2% of FBS, or uninfected. Cells were incubated for 1 h at 4° C. After incubation, cells were placed at 37° C. for 0, 10, 30 or 60 min. At each time, virus medium was removed and cells were washed three times with cold DPBS. Cells were collected in 350 μL of lysis buffer RA1 from NucleoSpin RNA kit as described above for RNA isolation and WNV genome copy number determination by RTqPCR.

### Transmission Electron Microscopy

Vero cells (10⁷) were infected with either WNV WT, M-A43G, M-I36F, M-I36F/A43G viruses at a MOI of 10 or uninfected. 24 h post-infection, cells were fixed for 24 h in 4% PFA and 1% glutaraldehyde (sigma) in 0.1 M phosphate buffer (pH 7.2). Cells were washed in PBS and post-fixed with 2% osmium tetroxide for 1 h. Cells were fully dehydrated in a graded series of ethanol solutions and propylene oxide. The impregnation step was performed with a mixture of (1:1) propylene oxide/Epon resin and left overnight in pure resin. Cells were then embedded in resin blocks, which were allowed to polymerize for 48 h at 60° C. Ultra-thin sections (70 nm—of blocks were obtained with a Leica EM UC7 ultramicrotome (Wetzlar). Sections were stained with 5% uranyl acetate and 5% lead citrate and observations were made with JEOL 1011 transmission electron microscope.

### Analysis of the Secreted Particles by Negative Staining Electron Microscopy and Immunogold Labeling

Viral particles from clarified cell culture were purified by polyethylene glycol precipitation followed by an ultracentrifugation at 50000G, 4° C. for 2 h (Ultracentrifuge Optima L-100 XP, Beckman) on iodixanol gradient (OptiPrep, Sigma-Aldrich). Fractions of interest were then collected and fixed (v/v) with paraformaldehyde (PFA) 2% (Sigma, St-Louis, Mo.), 0.1M phosphate buffer pH 7.2 for 24 h. Formvar/carbon-coated nickel grids were deposited on a drop of fixed sample during 5 min and rinsed three times with phosphate-buffered saline (PBS). After a single wash with distilled water, the negative staining was then performed with three consecutive contrasting steps using 2% uracyl acetate (Agar Scientific, Stansted, UK), before analysis under transmission electron microscope (JEOL 1011, Tokyo, Japan).

For immunogold labeling, grids coated with the sample were washed and further incubated for 45 min on a drop of PBS containing 1:10 mouse monoclonal antibody against Flavivirus E protein (4G2). After 6 washes with PBS, grids were incubated for 45 min on a drop of PBS containing 1:30 gold-conjugated (10 nm) goat-anti-mouse IgG (Aurion, Wageningen, Netherlands). Grids were then washed with 6 drops of PBS, post-fixed in 1% glutaraldehyde, rinsed with 2 drops of distilled water, before being negatively stained and observed under the microscope as described above.

### Ultrastructural Analysis of the Infected Cells by Transmission Electron Microscopy

24 h-infected Vero or C6/36 cells were trypsinized, rinsed once in PBS, and gently resuspended in cold fixation buffer containing paraformaldehyde 4% (Sigma, St-Louis, Mo.), 1% glutaraldehyde (Sigma), 0.1 M phosphate buffer pH 7.3, for 24 h. Cells were then placed in a mixture of (1:1) propylene oxide/Epon resin (Sigma) and left overnight in pure resin for samples impregnation. Cells were then embedded in Epon resin (Sigma), and blocks were allowed to polymerize for 48 hours at 60° C. Ultra-thin sections of blocks were obtained with a Leica EM UC7 ultramicrotome (Wetzlar, Germany). Sections were deposited on formvar/carbon-coated nickel grids and stained with 5% uranyl acetate (Agar Scientific), 5% lead citrate (Sigma), and observations were made with a JEOL 1011 transmission electron microscope.

### Mouse Experiments

Three-week-old female BALB/c mice were obtained from JANVIER LABS (France), housed under pathogen-free conditions in level 3 animal facility and protocols were approved by the Ethic Committee for Control of Experiments in Animals (CETEA) at the Institut Pasteur and declared to the French Ministry under no. 00762.02. Mice were inoculated intraperitoneally either with 50 FFU of either WNV WT, M-I36F, M-A43G or M-I36F/A43G mutant in 50 μL of DPBS supplemented with 0.2% bovine serum albumin or with DPBS alone as a negative control. Mice were monitored daily post-infection for onset of disease (weight loss, clinical symptoms and survival rate were followed). Blood samples were collected every 2 days pi by puncture at the caudal vein and tested for the presence of viral RNA. Mice that survived the infection were challenged with 1000 FFU of wild-type virus diluted in 50 μL of DPBS+0.2% BSA at day 28 pi. Mice mortality was followed over time. Blood was obtained by puncture at the caudal vein at day 27 pi, collected in tube containing EDTA and serum separated after centrifugation at 4000G, 10 min in order to perform ELISA and seroneutralization assays.

Direct ELISA

Viruses were purified by polyethylene glycol precipitation followed by utracentrifugation at 50000G, 4° C. for 2 h (Ultracentrifuge Optima L-100 XP, Beckman) on iodixanol gradient (OptiPrep, Sigma Aldrich). Fractions of interest were then UV-inactivated. High-binding 96-well plates (Nunc) were coated with 2 μg/mL of purified and inactivated viruses in 100 μL of PBS-3% milk and 0.5% Tween 20 (PBS-milk-Tween) and incubated overnight at 4° C. Plates were washed five times with PBS containing 0.05% Tween 20. mAb 4G2, polyclonal anti-WNV antibodies, or sera obtained from mice blood were serially diluted 10-fold (morphology analyses) or 2-fold (mice experiments) starting at 1:100 dilution in PBS-milk-Tween, added to plates and incubated 1 h at 41° C. After washing, plates were incubated with 100 μL of HRP-conjugated goat anti-mouse IgG diluted 1:10 000 in PBS-milk-Tween for 1 h at 41° C. Plates were washed again and 200 μL of SIGMAFAST™ OPD (Sigma) substrate was added per well for 30 min following manufacturer's instructions. Luminescence was read on plate reader EnVision™ 2100 Multilabel Reader (PerkinElmer, Santa Clara, Calif., USA) at a wavelength of 450 nm.

### Indirect ELISA

High-binding 96-well plates (Nunc) were coated with 5 μg/mL of polyclonal anti-WNV antibody in 100 μL of PBS-milk-Tween and incubated overnight at 4° C. Plates were washed five times with PBS containing 0.05% Tween 20 and 2 μg/mL of purified and inactivated viruses were added to plates and incubated 2 h at 41° C. After washing, 100 μL of HRP-conjugated mAb 4G2 serially diluted 10-fold in PBS-milk-Tween were added to plates and incubated 1 h at 41° C. Plates were washed again and 200 μL of HRP substrate, SIGMAFAST™ OPD (Sigma), was added per well for 30 min following manufacturer's instructions. Luminescence was read on plate reader EnVision™ 2100 Multilabel Reader (PerkinElmer, Santa Clara, Calif., USA) at a wavelength of 450 nm.

### Seroneutralization Assay

Mice sera were serially diluted (two-fold) in DMEM supplemented with 2% FBS, starting at dilution 1:20. Each dilution was incubated for 1 h at 37° C. with 500 FFU of WNV IS98 WT. The remaining infectivity was assessed by FFA on Vero cells as described above. Sera collected from mice inoculated with DBPS served as negative control. The 50% plaque reduction neutralization titer (PRNT50), corresponding to the serum dilutions at which plaque formation was reduced by 50% relative to that of virus not treated with serum, was calculated. Neutralization curves were obtained and analyzed using GraphPad Prism 6 software. Nonlinear regression fitting with sigmoidal dose response was used to determine the dilution of serum that reduced the quantity of FFU by 50%.

### Statistical Analysis

Data were analyzed with Prism 6 Software (GRAPHPAD software). Titers and RNA quantitation were evaluated for statistically significant differences by non-parametric Mann-Whitney test. Survival proportions were evaluated for statistically significant differences by log-rank (MANTEL-COX) test.

### Example 2 (ZIKV)

### Materials and Methods

### Cells

Human neuroblastoma cells SK-N-SH (ATCC® HTB-11™) and simian kidney cells Vero (ATCC® CRL-81™) were cultured at 37° C. in Dulbecco's Modified Eagle Medium (DMEM, INVITROGEN) supplemented with 10% of fetal bovine serum (FBS).

Aedes albopictus cells C6/36 [ATCC® CRL-1660™] were cultured at 28° C. in Leibovitz's 15 (L15, INVITROGEN) medium containing 10% of FBS and 1% of penicillin and streptomycin.

### Generation of I36F, A43G and I36F/A43G ZIKV Mutants

Any infectious clone of ZIKV (i.e. any plasmid backbone comprising a full length ZIKV genome) can be used to introduce the I36F, A43G or I36F/A43G mutations in the genome. The inventors used a construct containing the genome of ZIKV strain MR766, and performed site-directed mutagenesis by PCR using PHUSION High Fidelity (Thermo Fischer Scientific) employing the following primers to introduce the mutations M-I36F and M-A43G respectively: FW (M-I36F): 5′-GGTTGAAAACTGGTTTTTCAGGAACCCC-3′ (SEQ ID NO: 33), RV (M-I36F): 5′-GGGGTTCCTGAAAAACCAGTTTTCAACC-3′ (SEQ ID NO: 34) and FW (M-A43G): 5′-AACCCCGGGTTTGGACTAGTGGCCGTT-3′ (SEQ ID NO: 35), RV (M-A43G): 5′-AACGCCACTAGTCCAAACCCGGGGTT-3′ (SEQ ID NO: 36). WT or mutant ZIKV virions were obtained after transfection of 2.5 μg of each construct in mosquito cells C6/36 using Lipofectamine 3000 (Thermo Fischer Scientific). Viral stocks were then amplified in mosquito cells.

### Infections

Virus infections were performed in 24-well-culture plaques. 10⁵ SK-N-SH cells or VERO cells were seeded. 24 hours later, they were infected with 200 μL of medium containing a given number of viral particles, depending on the MOI. One hour after inoculation, inoculum was replaced by medium containing 2% of FBS.

Titration in ffu/mL

Vero-NK cells were seeded at 8×10⁴ cells per well in 24-well plates and incubated at 37° C. for 24 h. Ten-fold dilutions of virus in DMEM were added to the cells and incubated for 1 h at 37° C. Unabsorbed virus was removed, then 1 ml of DMEM supplemented with 1.6% carboxymethyl cellulose (CMC), 10 mM HEPES buffer, 72 mM sodium bicarbonate, and 2% FBS was added to each well, followed by incubation at 37° C. for 3 days. The CMC overlay was removed, the cells were washed with PBS and fixed with 4% paraformaldehyde for 15 min, followed by permeabilization with 0.2% Triton X-100 for 5 min. Cells were then washed with PBS and incubated for 1 h at room temperature (RT) with anti-E antibody (4G2), followed by incubation with HRP-conjugated anti-mouse IgG antibody. The foci were revealed using the Vector VIP peroxidase substrate kit (Vector Laboratories; catalog no. SK-4600) according to the manufacturer's instructions.

### Quantitative RT-PCR

Total RNA were extracted from samples as described for WNV (see Example 1). The RNA standard used for quantification of ZIKV copy numbers was in vitro transcribed from a Sall-linearized ZIKV-NS5 plasmid. In vitro transcribed RNA were synthetized using the MEGAscript SP6 transcription kit (Life technologies) according to manufacturer's instructions. The quantitation of a given target RNA was performed using 2 μl of RNA and the SYBR green PCR Master Mix kit (ThermoFisher Scientific; catalog no. 4344463) according to manufacturer's instructions. The real-time PCR system (Thermofisher scientific) was used to measure SYBR green fluorescence with the same program as for WNV. Primers 5′-ATGGAAGACGGCTGTGGAAG-3′ (SEQ ID NO: 37) and 5′-GCTCCCAACCACATGTACCA-3′ (SEQ ID NO: 38) were used for viral genome quantification. Target gene expression was normalized to the expression of GAPDH mRNA, measured using the 2 primers 5′-GGTCGGAGTCAACGGATTTG-3′ (SEQ ID NO: 14) and 5′-ACTCCACGACGTACTCAGCG-3′ (SEQ ID NO: 15).

### Transmission Electron Microscopy:

Vero cells (1.10⁷) were infected with either WNV WT, M-A43G, M-I36F, M-I36F/A43G viruses at a MOI of 10 or uninfected. 24 h post-infection, cells were fixed for 24 h in 4% PFA and 1% glutaraldehyde (Sigma) in 0.1 M phosphate buffer (pH 7.2). Cells were washed in PBS and post-fixed with 2% osmium tetroxide for 1 h. Cells were fully dehydrated in a graded series of ethanol solutions and propylene oxide. The impregnation step was performed with a mixture of (1:1) propylene oxide/Epon resin and left overnight in pure resin. Cells were then embedded in resin blocks, which were allowed to polymerize for 48 h at 60° C. Ultra-thin sections (70 nm of blocks were obtained with a Leica EM UC7 ultramicrotome (Wetzlar). Sections were stained with 5% uranyl acetate and 5% lead citrate and observations were made with JEOL 1011 transmission electron microscope.

### Statistical Analysis

Data were analyzed with Prism 6 Software (GRAPHPAD software). Titers and RNA quantification were evaluated for statistically significant differences by non-parametric t-tests.

### Results

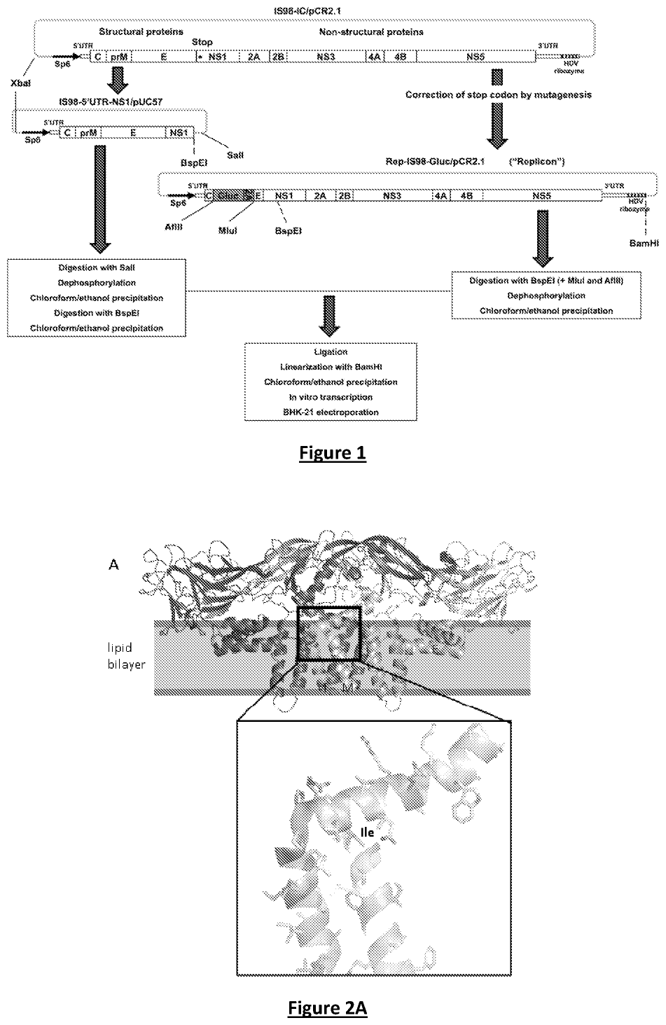

### FIG. 1: Schematic Representation of IS98 Viral Production Using the Two-Plasmid Infectious Clone Technique.

WNV IS98 infectious clone was divided into two plasmids. Rep-IS98-Gluc/pCR2.1 is a replicon that contains the non secreted form of Gaussia Luciferase instead of the structural genes. IS98-5′UTR-NS1/pUC57 contains a copy of the genome region comprised between 5′UTR and the N-terminus of NS1 under the control of a SP6 promotor that encompasses the structural region of the virus genome. Both plasmids are digested, ligated linearized and in vitro transcribed to produce full length viral RNA.

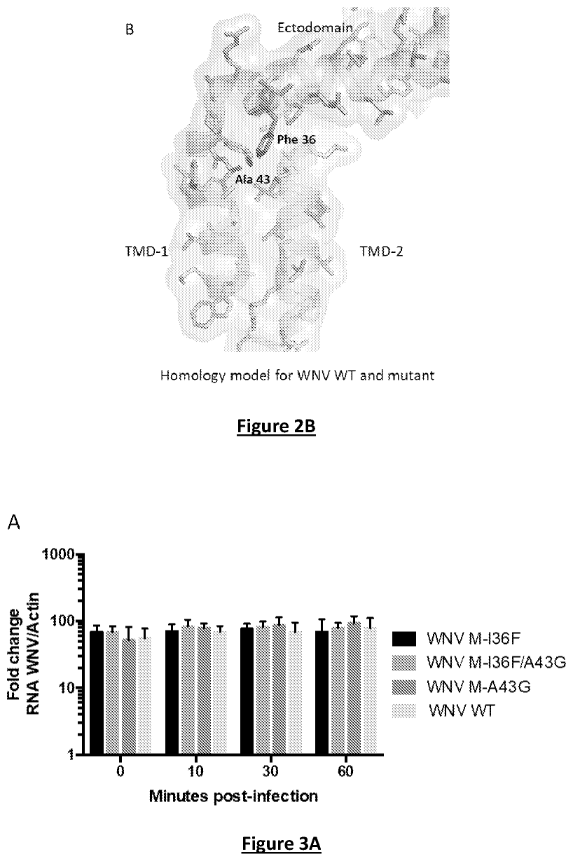

### FIGS. 2A-2B: WNV M Protein Structural Analysis and M-I36F Mutation

2A. JEV M protein crystallized structure (ref 2016).

2B. Homology model for WNV M protein WT and mutant. Substitution of isoleucine (Ile) 36 with phenylalanine (Phe) caused a putative clash (negative interaction) with the side chain of the alanine (Ala) 43 (M-A43) of the same protein, located in the transmembrane domain 1 (TM-1) and directly in front of the mutated amino-acid in the 3-dimensional fold of the polypeptide. To relieve the clash caused by the first mutation the residue M-A43 was substituting with a glycine (Gly) (M-A43G) which has no side chain.

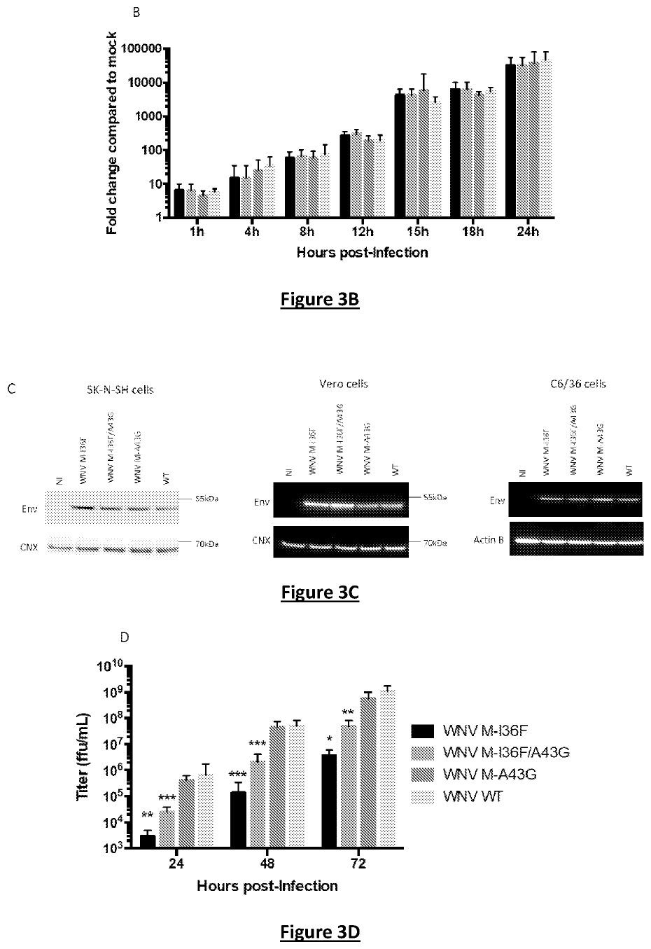

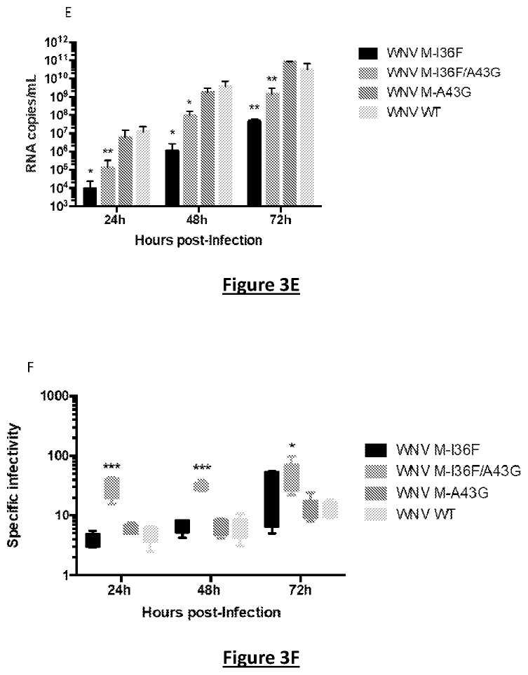

### FIGS. 3A-3F: M-I36F and M-I36F/A43G Mutations Affected WNV Cycle in Mammalian Cell

WNV is an arbovirus that infects both mosquitoes and mammals. In a first time, viruses were produced in Aedes albopictus C6/36 cells and the stability of the mutation was confirmed by Sanger sequencing. To investigate the effect of mutations on virus infectious cycle, human neuroblastoma-derived cells (SK-N-SH) were infected with either WNV WT, WNV M-A43G, M-I36F or M-I36F/A43G or uninfected.

3A. To determine whether WNV mutants were able to infect cells, we exposed SK-N-SH cells to WNV WT, WNV M-A43G, WNV M-I36F and WNV M-I36F/A43G at an MOI of 10 and carried out a single-hit infection assay at early time points of infection. Genomic viral RNA were detected without difference between WT and mutants viruses suggesting that mutants attached to cell membrane and penetrated into it similarly to the WT.

3B. Viral replication capacity was assessed by performing a time course infection and tested for the amplification of viral RNA. A similar pattern of viral RNA amplification was observed between the four viruses up to 24 h pi demonstrating that M mutations did not affect viral replication over time.

3C. Cell lysates from infected SK-N-SH, Vero or C6/36 cells (MOI=1) were collected 24 h pi and viral protein synthesis was tested by Western Blot. A slight increase in viral envelope protein was observed in Vero and SK-N-SH cell lysate infected with WNV M-I36F or WNV M-I36F/A43G as compared to WT and M-A43G viruses, indicating that WNV mutants protein synthesis is not affected by the mutations, but strongly suggest an accumulation of viral protein. No significant difference in viral protein accumulation was observed 24 h pi in C6/36 cells showing that WNV mutants protein synthesis is comparable to WT.

3D. Supernatants from infected SK-N-SH cells (MOI=1) were harvested at 24 h, 48 h and 72 h pi and infectious particles production was quantify. A decrease by around 2.5 logs and 1.8 logs in titers was observed in the supernatants of cells infected with WNV M-I36F and M-I36F/A43G respectively as compared to WT. Interestingly, WNV M-A43G produced as many infectious particles as WT, showing that the M-A43G mutation alone did not affect WNV cycle.

3E. In addition, viral genomic RNA extracted from the same supernatants were measured by qRT-PCR. Less viral RNA were observed in the supernatants of cells infected with WNV M-I36F (3.1 logs) and WNV M-I36F/A43G (2 logs) indicating that lower infectious particles were released from SK-N-SH cells infected with either WNV M-I36F or WNV M-I36F/A43G as compared to WT.

3F. Relative specific infectivity of each virus was measured as a ratio of WNV RNA to infectious particles. Specific infectivity of WNV M-I36F/A43G was overall higher than that of WT implying that a certain level of non-infectious particles was produced when SK-N-SH cells were infected with WNV M-I36F/A43G. More importantly, this observation strongly suggests that M-I36F/A43G mutations together might alter viral morphogenesis. Same experiments were performed using Vero cells and similar results were obtained. Statistical analyses performed using Student's t-test *: P<0.1; **: P<0.01; ***: P<0.001

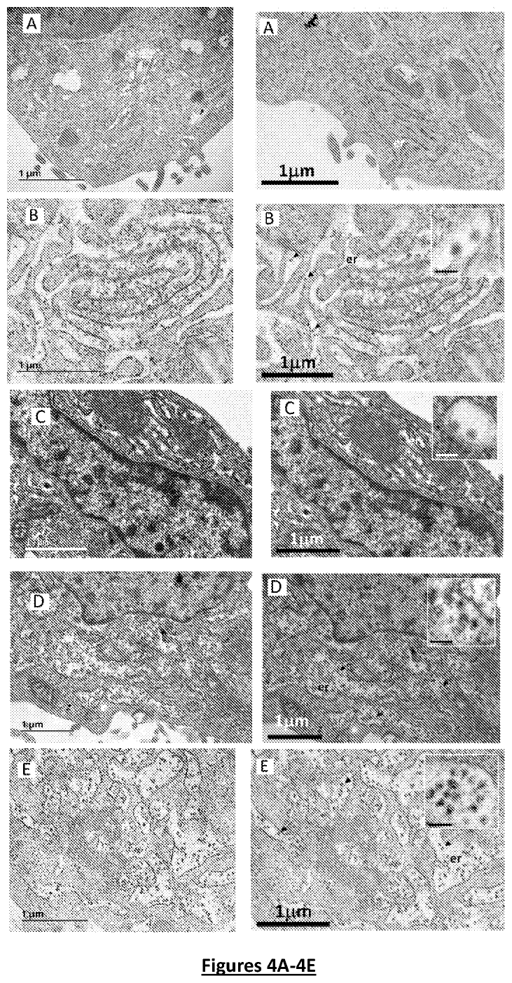

### FIGS. 4A-4E: M Protein Mutations Lead to Extensive Viral Particles Retention

To provide ultrastructural details, transmission electron microscopy of cells infected with either WNV WT, M-I36F, M-A43G, M-I36F/A43G (MOI=10) or uninfected was performed.

4A. Non infected VERO cells displayed normal morphology and nuclear membrane integrity 24 h pi.

4B. In contrast, WT-infected cells presented an electron dense perinuclear region with numerous convoluted membranes containing viral particles that likely corresponds to viral factories, the primary sites of viral production.

4C. As WT-infected cells, WNV M-A43G virus induced abundant membrane rearrangements in the perinuclear region and viral particles are observed in the endoplasmic reticulum (ER) or ER-derived vesicles.

4D. Significantly larger ER-vacuoles were observed throughout cell cytoplasm when cells were infected with WNV M-I36F mutant. These vacuoles contained many viral particles within their lumen, indicating that they are major sites of virions accumulation.

4E. Like WNV M-I36F infection, cells infected with WNV M-I36F/A43G displayed cytoplasmic vacuolization and important viral particles accumulation in ER and ER-derived vesicles suggestion that M-I36F mutation impairs viral egress and underlying the importance of M protein in viral secretion.

Importantly, the M-I36F and M-I36F/A43G mutant particles were released into the ER lumen of the infected mammalian cells and not retained at the ER membrane indicating that assembly and budding steps still occurred in the presence of the M-I36F mutation alone or associated with M-A43G (cf. zooms).

### M-I36F Mutation Strongly Inhibits WNV Efficient Secretion.

As only a few M-I36F/A43G and M-I36F mutant particles were found in the supernatant of mammalian cells, the inventors wondered whether the M-I36F mutation could interfere with proper budding and/or secretion of the viral particles. The inventors examined mammalian cells infected with the different viruses by electron microscopy (FIGS. 4A-4E). Specific sub-cellular ultrastructural changes associated with the presence of each virus were observed in ultrathin sections of Vero cells infected with either wild-type or mutant viruses (FIG. 4, panels A, B, C and D). Relatively few wild-type and M-A43G viral particles were observed within the cells, with the occasional particle found in the ER, indicating that the virions are secreted normally (FIGS. 4A and 4B, arrows). On the other hand, in the same cell type, infection with the M-I36F or M-I36F/A43G virus induced large ER swelling and massive accumulation of newly formed viral particles within the ER and ER-derived vesicles (FIGS. 4C and 4D, arrows). No such impairment of particle secretion with either mutant was observed in infected mosquito cells (FIG. 12). Importantly, the M-I36F and M-I36F/A43G mutant particles were released into the ER lumen of the infected mammalian cells and not retained at the ER membrane indicating that assembly and budding steps still occurred in the presence of the M-I36F mutation alone or associated with M-A43G (FIGS. 4A, 4B, 4C and 4D, zooms). The overall aspect of WNV M-I36F and M-I36F/A43G mutant particles seemed irregular as compared to wild-type and M-A43G mutant viruses in mammalian cells (FIGS. 4A, 4B, 4C and 4D, zooms), suggesting that WNV morphology was potentially altered by the M-I36F mutation. Indeed, the few secreted M-I36F/A43G virions into the supernatant of mammalian cells at 24 h pi directly observed by standard negative staining electron microscopy seemed to display an altered morphology although the nucleocapsid and the lipid envelope were still well delineated (FIG. 13C). While the inventors were unable to obtain any image for M-I36F mutant due to an insufficient number of secreted particles, that of wild-type and mutant M-A43G virions showed typical characteristics of flavivirus particles (FIGS. 13A and 13B). On the other hand, wild-type and mutant M-I36F, M-A43G and M-I36F/A43G virions collected from supernatants of mosquito cells all displayed the morphological characteristics of classic flaviviruses (FIG. 14). The specificity of the observed particles was confirmed using immunogold labeling with mAb 4G2 and the presence of WNV E protein at the surface of wild-type, M-A43G or M-I36F/A43G virions was unambiguously observed (FIGS. 13D, 13E and 13F), although less labeling was found at the surface of the double mutant virions. Taken together these data suggest that the M-I36F mutation affects virion secretion possibly by altering WNV morphology only in mammalian cells.

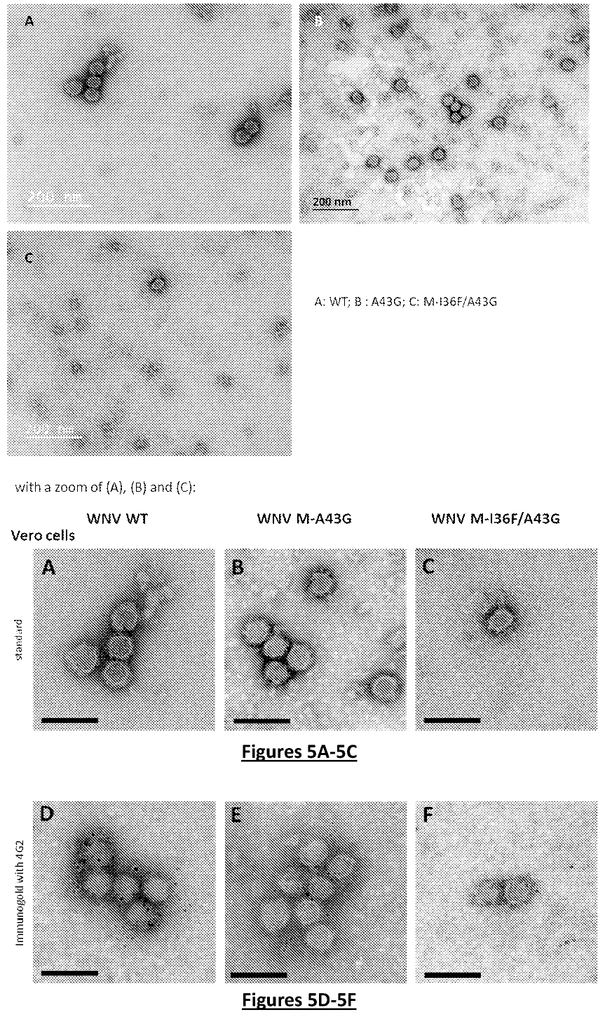

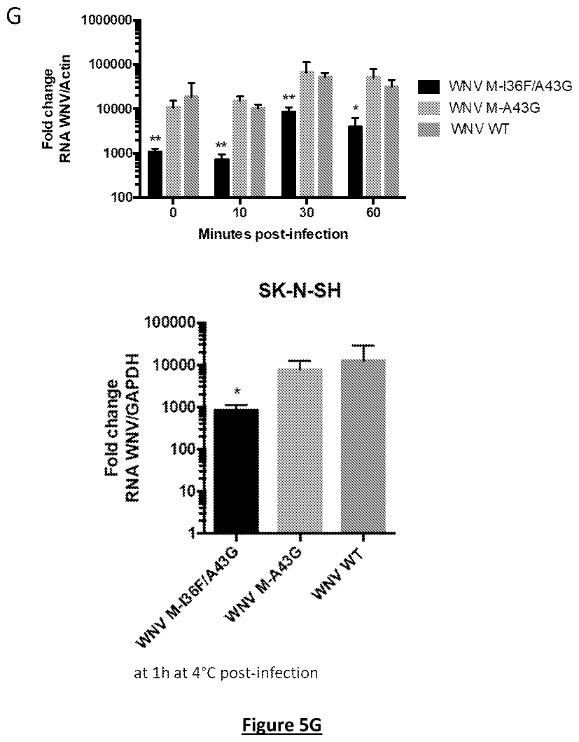

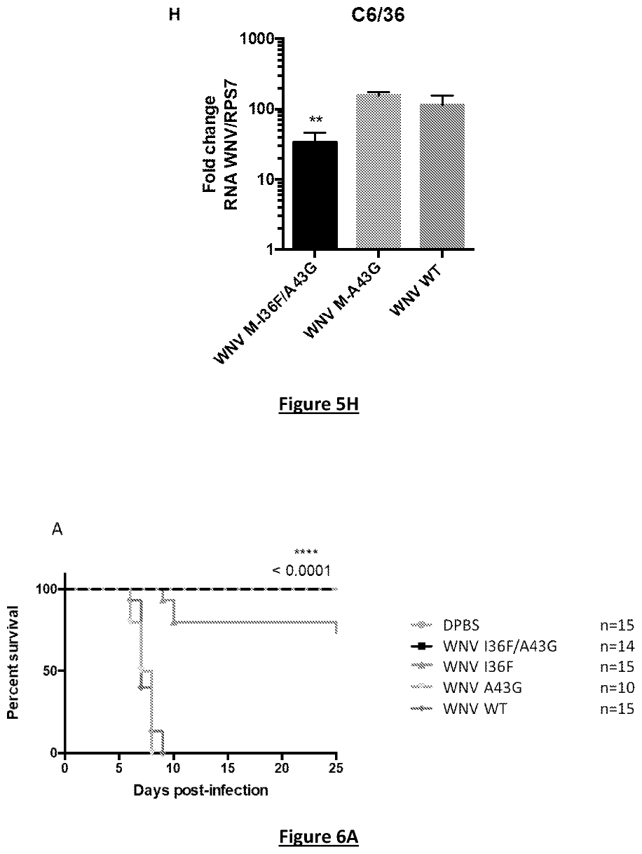

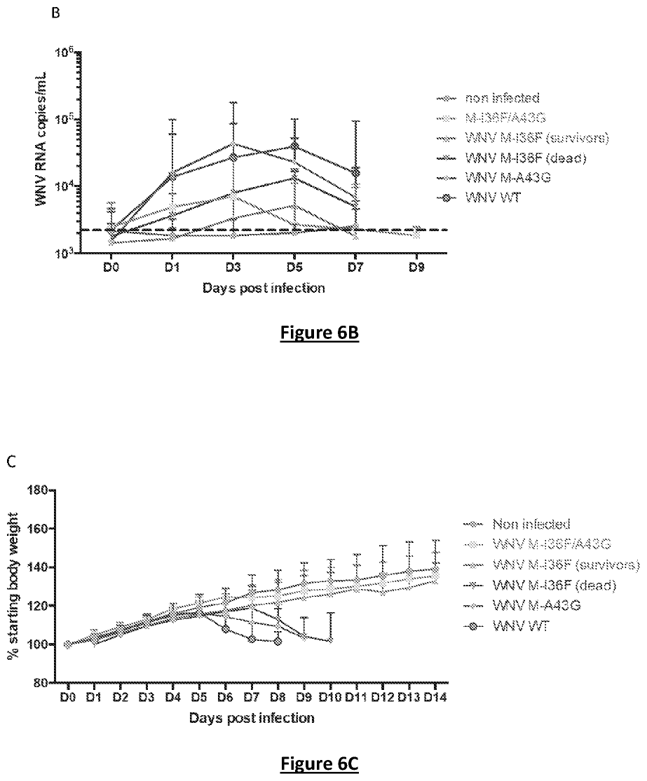

### FIGS. 5A-5D: Mutations in the M Protein Modify Viral Particles Morphology and Lead to Defective Particles Production

To analyse secreted viral particles morphology, negative staining electron microscopy of purified and concentrated culture supernatant from VERO cells infected with either WNV WT, WNV M-A43G or WNV M-I36F/A43G viruses was performed. Amount of WNV M-I36F viral particles released in the supernatant was too small to be visualized in electron microscopy.

5A (+ zoom). WT viruses presented numerous spherical particles, 50 to 60 nm of diameter that had morphological characteristics of a typical flavivirus.

5B (+ zoom). As WT, WNV M-A43G viruses displayed expected classical flavivirus morphology. Nucleocapsid (dark centre) is surrounded by the lipid envelope (pale halo) in which envelope and membrane proteins are inserted.

5C (+ zoom). In contrast, WNV M-I36F/A43G viruses presented a very heterogenous morphology with many non-spherical particles, demonstrating that introduction of M-I36F mutation in the M protein of WNV impaired its morphogenesis.

5D, 5E, 5F. The specificity of the observed particles was confirmed using immunogold labeling with Mab 4G2 and the presence of WNV E protein at the surface of wild-type, M-A43G or M-I36F/A43G virions was unambiguously observed, although less labeling was found at the surface of the double mutant virions.

5G. SK-N-SH cells were infected with viruses produced from VERO cells and viral infectivity of WNV WT, WNV M-A43G and WNV M-I36F/A43G were investigated. Cells were exposed to viruses at an MOI of 10 and viral RNA attached to the cells were quantify by qRT-PCR at early time points of infection. WNV M-I36F/A43G genomic viral RNA attached to the cell surface (0 min pi) were decreased by around 1.2 logs as compared to WT and M-A43G viruses suggesting that modification of viral morphology impairs WNV M-I36F/A43G infectivity.

5H: Same as 5F, with C6/36 cells.

### FIGS. 6A-6E: Alteration of WNV Particles Morphology Leads to Viral Attenuation in a Mouse Model

6A. Three-week-old BALB/C female mice were infected intraperitoneally with 50 FFU of WNV WT or with the different mutant viruses. Survival percentages were calculated (****, P<0.0001, LogRank test). All the mice infected with WNV M-I36F/A43G survived to the infection while only 66% of mice infected with WNV M-I36F survived and none of them resisted to the infection with WNV M-A43G and WT, underlying that the introduction of both mutations is essential for viral attenuation.

6B. Blood samples were collected at days 0, 1, 3, 5, 7 and 9 pi and viremia developed by mice was tested by qRT-PCR. WT virus is detected in mice blood from 24 h pi, reached a peak at day 5 pi, and then decreased. As WT, mutant M-A43G virus is detected in mice blood from 24 h pi, but peak of viremia is observed earlier (day 3 pi). M-I36F mutant virus disseminated later in mice blood since virus was detected only from day 3 pi and reached a peak at day 5 pi. Viral load is lower than that of WT. WNV M-I36F/A43G mutant is detected in blood from 24 h pi as WT and WNV M-A43G viruses. However, viral load is decreased by around 1 log overtime as compared to WT and M-A43G viruses and peak of viremia is observed at 3 day pi.

6C. Mice growth was followed up to 14 days post inoculation by measuring body weight every day. Infection with WNV WT or WNV M-A43G led to a significant weight loss from 7 days pi that correlated with disease development. While the growth of mice infected with WNV M-I36F was heterogenous among the group, a growth delay was observed from day 7 pi reflecting that 5 mice over 15 got sick and died from the infection. However, the global weight loss is lower than that of WT and M-A43G viruses. Mice inoculated with WNV M-I36F/A43G virus presented a growth curve similar to the one of non-infected mice, showing that WNV M-I36F-A43G is fully attenuated in vivo.

6D. Mice were challenged with 1000 FFU of WNV WT at 28 days pi. All the mice that survived the first infection with either WNV M-I36F or WNV M-I36F/A43G mutants, resisted to the lethal challenge, while all the noninfected mice died from the infection. This shows that the immune response developed by mice primary infected with WNV M-I36F/A43G is important enough to protect them against WNV WT.

6E, 6F. Sera were collected 27 days after inoculation from the mice that survived the infection and the presence of WNV specific-IgG and neutralizing antibodies were measured by ELISA and PRNT₅₀ respectively. A single intraperitoneal injection of either M-I36F or M-I36F/A43G into adult BALB/c mice induced high levels of both WNV-specific IgG and neutralizing antibodies (geometric mean titer=102.86, and 110 respectively), as compared to sera collected from non infected mice. Seroneutralizing assay was performed on dilutions using virulent WT virus as target. Neutralizing antibody against WNV WT were largely produced by mice that survived infection with either WNV M-I36F or WNV M-I36F/A43G (PRNT₅₀>640) as compared to sera collected from non infected mice.

FIG. 7: Alignment of M protein sequences from flaviviruses. The isoleucine at position 36 of the WNV M protein is conserved in Dengue virus 4, JEV, WNV, and Zika virus. The alanine at position 43 of the WNV M protein is conserved in Dengue virus 4, JEV, WNV, and Zika virus.

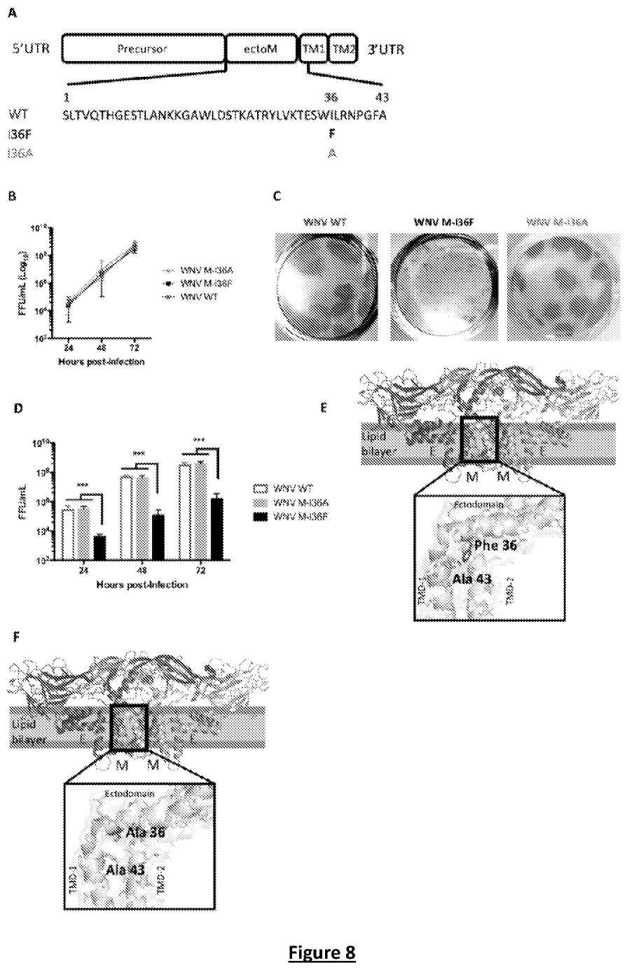

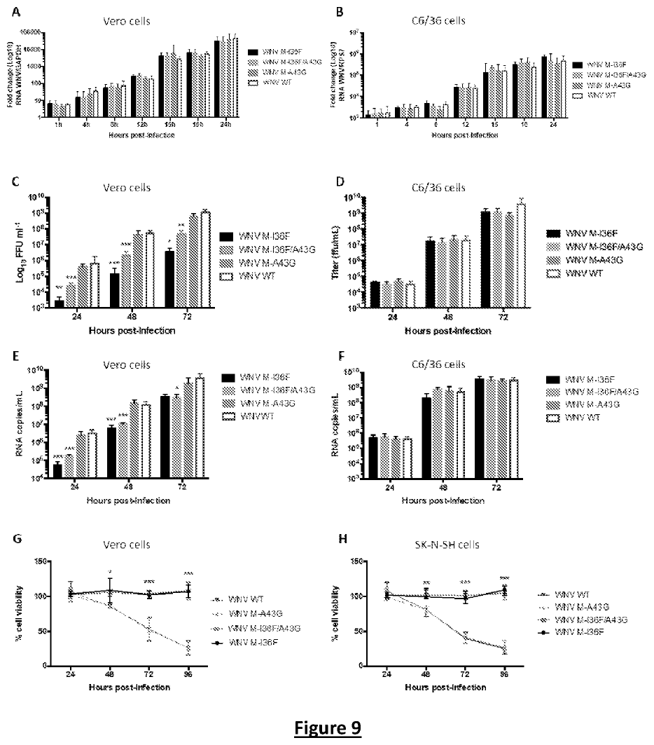

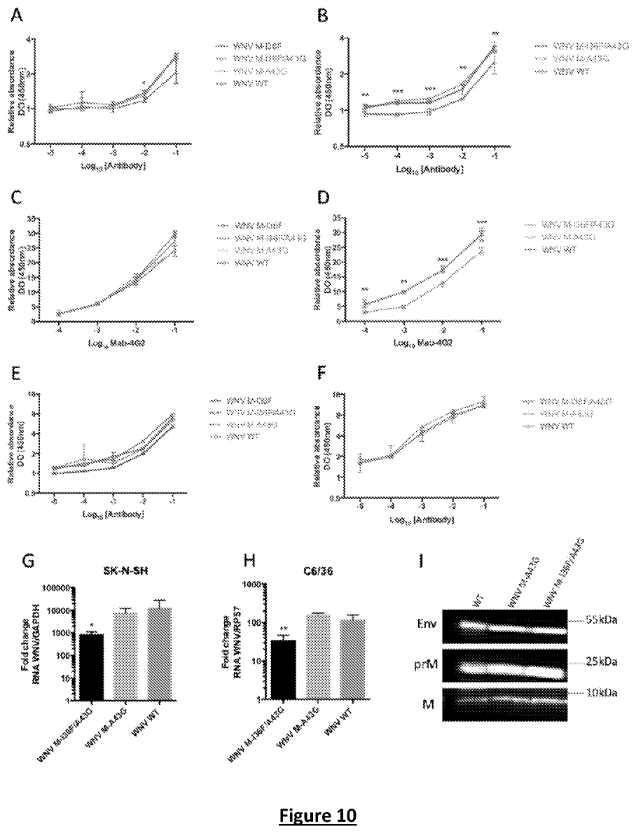

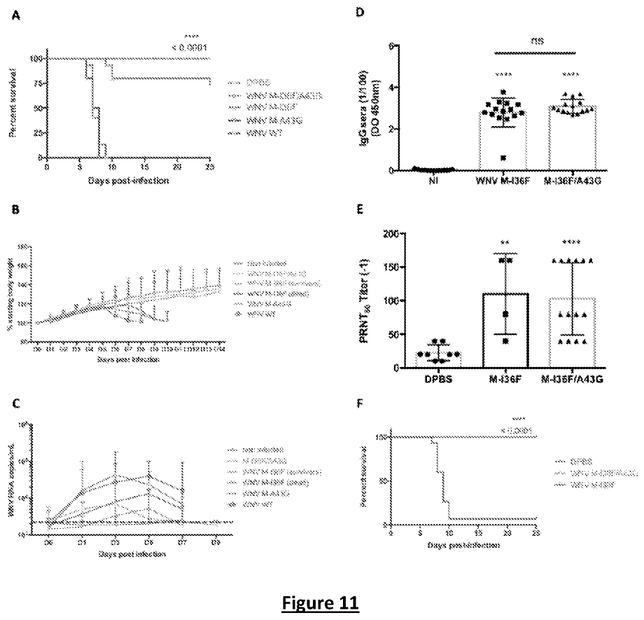

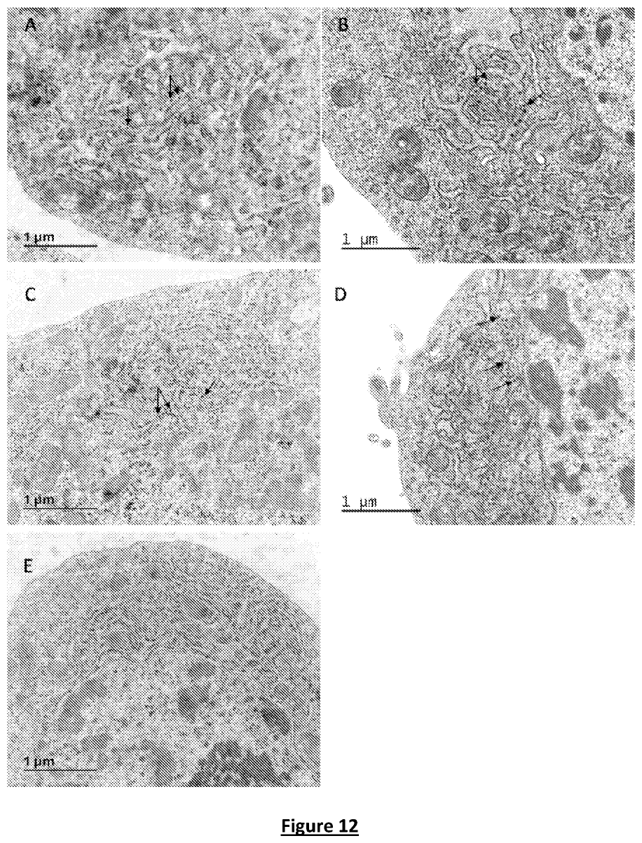

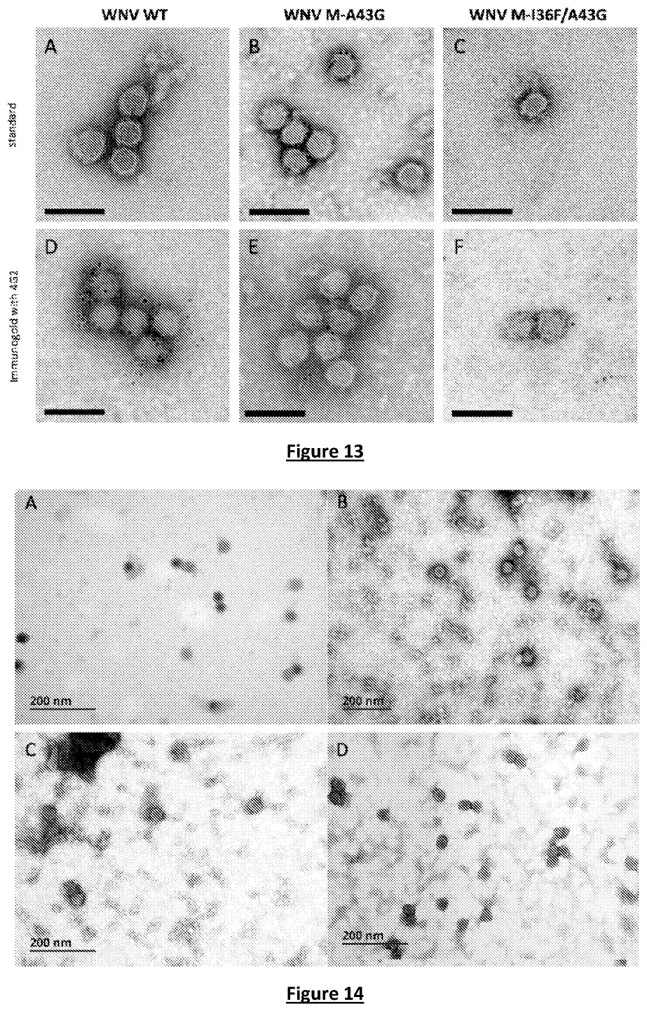

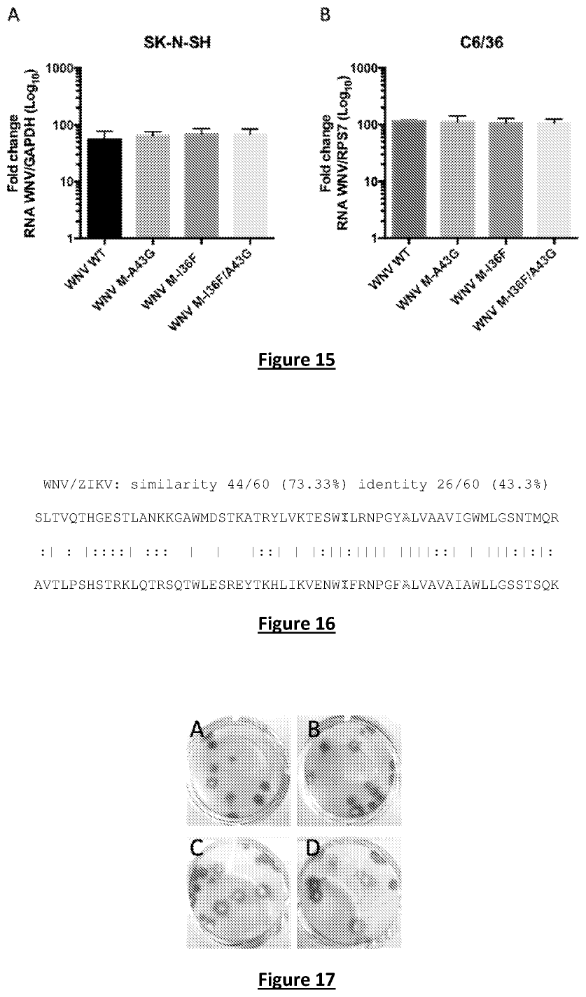

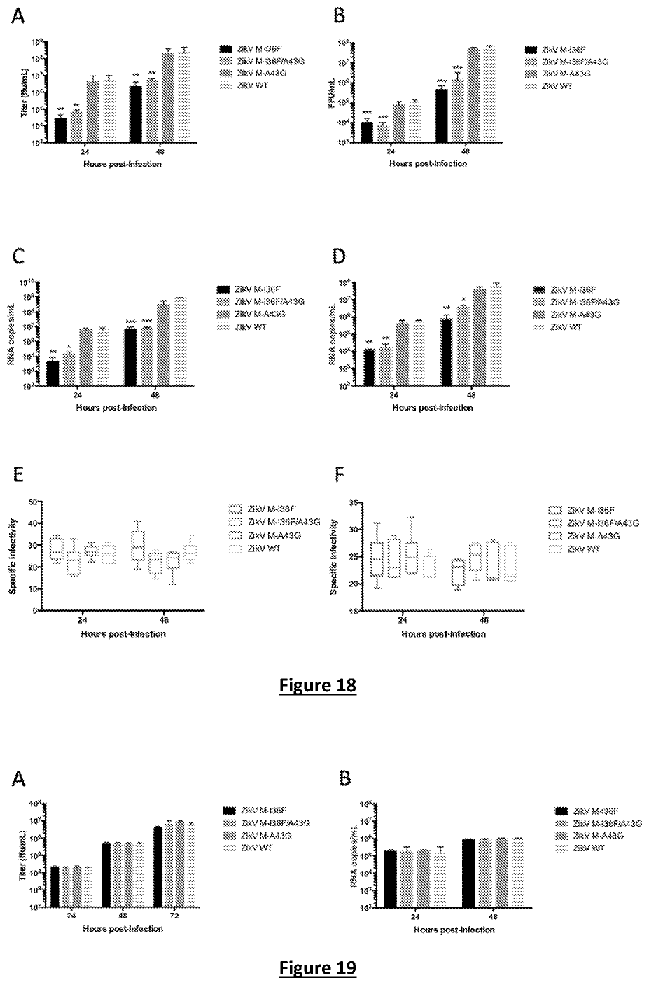

FIG. 8: Mutation of M-36 affects WNV infectious cycle by potentially altering the M protein 3-dimensional structure. Mirroring the M-L36F mutation in the YFV 17D vaccine, the inventors replaced isoleucine 36 of the WNV M protein with a phenylalanine (M-I36F) (FIG. 8A). The resulting mutant virus was successfully produced in C6/36 cells electroporated with genomic RNA synthesized in vitro (see Material and Methods) (FIG. 8B) and contrary to wild-type WNV, M-I36F mutant displayed a smaller foci phenotype in Vero cells, which is a potential attenuation marker (FIG. 8C, M-I36F). Conversely, substitution of the parental isoleucine 36 with an alanine (FIG. 8A, M-I36A) did not affect foci size (FIG. 8C, M-I36A). More importantly, the inventors observed that only the M-I36F mutation impaired WNV infectious cycle in mammalian neuronal SK-N-SH cells (FIG. 8D) suggesting that the nature of residue 36 is essential for efficient viral particle production in these cells. While isoleucine, alanine and phenylalanine possess close chemical properties, only the phenylalanine has an aromatic ring. To examine how M-I36F might physically impact interactions with neighboring amino acids, the inventors mapped either WNV M-I36F or WNV M-I36A into the recently published JEV M protein 4.3 Å cryo-EM structure (Wang et al., 2017, Nat Commun 8:14) revealing that M-I36F (FIG. 8E), but not M-I36A (FIG. 8F), clashes with an alanine residue (A43) located in the first transmembrane segment of M (TM1). Thus, while interactions between the two structural proteins E and M are seemingly conserved, M-I36F potentially disrupts the M protein 3-dimensional structure such that steric hindrance is introduced between the phenylalanine aromatic ring and the side chain methyl group of A43.

### FIG. 9. Compensatory Mutation Partially Rescues M-I36F Mutant to Wild-Type Phenotype.

To compensate the potential clash between the aromatic ring of residue 36 and the side chain of residue 43, the inventors substituted the original A43 by a residue that has no methyl group, namely a glycine (M-A43G) in order to create more space, thereby generating a double mutant virus M-I36F/A43G. The inventors recovered and amplified WNV M-I36F/A43G, M-A43G and wild-type viruses from mosquito C6/36 cells electroporated with genomic RNA synthesized in vitro (see Material and Methods). All viruses were found to form large foci on mammalian Vero cells (data not shown), and replicated similarly as assayed for RNA production, in Vero (FIG. 9A) and C6/36 cells (FIG. 9B), indicating that the M-I36F and M-A43G mutations alone or together did not affect genome decapsidation and replication in mammalian and mosquito cells. When comparing infectious particle production in Vero cell supernatants, however, the titers of M-I36F as well as M-I36F/A43G variants were largely lower than that of wild-type and M-A43G viruses (1.42 logs and 0.93 logs respectively, FIG. 9C). Yet the M-I36F/A43G titers were significantly higher than that of M-I36F (FIG. 9C). Interestingly, when viruses were grown in C6/36 cells, no difference in titers was observed (FIG. 9D). Genetic stability of the mutant viruses was tested by 10 serial passages in Vero cells. Full-genome analysis of M-I36F/A43G passaged up to 10 times revealed the presence of both M-I36F and M-A43G and no other mutation along the genome, while M-I36F alone had already reverted to WT sequence at passage 2 without compensatory mutation elsewhere in the genome (data not shown). A decrease in the amount of genomic viral RNA was observed over time in Vero cell supernatants infected with M-I36F or M-I36F/A43G mutants as compared to wild-type or M-A43G viruses (FIG. 9E), that mirrored the decrease in infectious titers in mammalian cells (FIG. 9C) and corroborating a decrease in the number of secreted particles. No change in the amount of genomic viral RNA in mosquito cells infected either with wild-type or any mutant viruses was detected (FIG. 9F), again reflecting what the inventors observed in terms of titers in these cells (FIG. 9D). Interestingly, neither M-I36F nor M-I36F/A43G mutant virus infection of mammalian cells induced any cell death (FIG. 9G, Vero cells, and 9H, SK-N-SH cells), contrary to WT and A43G viruses. This result agrees with previous reports showing that residue M-36 can modulate the death-promoting activity of the M protein ectodomain of Flaviviruses (Brabant M, et al. 2009. Apoptosis 14:1190-1203, Catteau A, et al., 2003, J Gen Virol 84:2781-2793). No cell death induction was observed for either WT or any mutant viruses in infected C6/36 mosquito cells (data not shown). Altogether, these results indicated that the M-I36F mutation leads to an impaired WNV infectious cycle in mammalian cells, most likely due to the alteration of mutant viral assembly and/or egress, that can be partially rescued and completely stabilized by introduction of a second mutation relieving steric hindrance (M-A43G).

FIG. 10. Atypical Particle Morphology of the M-I36F/A43G Variant Impacts WNV Antigenic Profile.

Thus potential modification(s) of M protein structure caused by the M-I36F might lead to altered viral particle morphology with irregularly shaped mutant virions. The inventors reasoned that such atypical morphology of the mutant particles may impact the virion antibody recognition. The inventors therefore first evaluated the recognition profile of wild-type and mutant virions by direct ELISA (FIG. 10). While viruses produced in C6/36 cell supernatants are all similarly recognized by the mAb 4G2 that binds specifically the fusion loop of the E protein (Crill W D, Chang G-JJ. 2004. J Virol 78:13975-13986; Summers P L, et al. 1989. Virus Research 12:383-392) (FIG. 10A), recognition of M-I36F/A43G virus collected in the supernatant of Vero cell is significantly decreased by approximately 1.2-fold for any antibody dilution when compared to wild-type and the M-A43G (FIG. 10B). A similar significant decrease (ranging from 1.2 to 2-fold, depending on the antibody dilution) in recognition of WNV M-I36F/A43G produced in Vero cells by mAb 4G2 was obtained using indirect non-competitive ELISA (FIGS. 10C and 10D). Importantly, no difference in recognition by polyclonal anti-WNV antibody of wild-type and mutant viruses produced either in insect (FIG. 10E) or mammalian cells (FIG. 10F) was observed, indicating that despite a slight significant decreased recognition of protein E fusion loop, the general antigenic properties of WNV M-I36F/A43G mutant virus are conserved.

WNV surface epitopes are essential for both efficient recognition and cell attachment, and the proper folding of the E protein chaperoned by the M protein in the prM-E complex plays a critical role in them. The inventors therefore tested the infectious capacity of our mutant and wild-type viruses under conditions allowing viral binding, but not internalization, to SK-N-SH mammalian cells or C6/36 mosquito cells by evaluating viral genomic RNA associated with the cell surface (FIGS. 10G and 10H respectively). Comparing viruses produced in mammalian cells and assayed at the surface of SK-N-SH or C6/36 cells, levels of M-I36F/A43G RNA were reduced by around 1-log as compared to that of the wild-type and M-A43G viruses (FIGS. 10G and 10H), indicating that the WNV double mutant M-I36F/A43G has impaired binding to host cells. Conversely, wild-type, M-I36F, M-A43G and M-I36F/A43G produced in insect cells showed no difference in RNA levels (FIGS. 15A and 15B). To confirm that the decreased infectious capacity of M-I36F/A43G mutant virus was not simply due to a lack of maturation, the inventors tested for the presence of immature (prM) and mature (M) forms of the membrane glycoprotein at the surface of wild-type or mutant virions collected from Vero cell supernatants by Western Blot (FIG. 101). The presence of similar levels of prM and M for wild-type and mutant viruses alike suggested that all viruses undergo a similar maturation process. Taken together, the above results support the notion that virions harboring the M-I36F mutation have a possible altered morphology, while the main WNV antigenic properties are conserved.

FIG. 11. In Vivo Effects of WNV M-I36F and/or M-A43G Mutations.

The in vitro properties of WNV M-I36F and M-I36F/A43G mutants encouraged the inventors to test their phenotype in vivo. The inventors first assessed pathogenicity in a well-established mouse model of WNV-induced encephalitis (Lucas M, et al. 2004. Virology Journal 1:9-9). In contrast to the high mortality rate observed among mice infected with either wild-type or M-A43G WNV (in which all 15 animals died), only 4 of 15 WNV M-I36F-infected mice died after being infected while all mice infected with M-I36F/A43G survived (FIG. 11A). As expected, the wild-type, M-A43G and M-I36F-infected mice that died presented rapid weight loss beginning at day 6 pi (FIG. 11B). Conversely, rather than weight loss, the inventors observed normal weight gain among mice that survived the infection (FIG. 11B). To investigate whether WNV M-I36F and M-I36F/A43G mutants were attenuated due to a less effective viral dissemination, the inventors collected blood samples every other day following the infection, for 10 days, and assayed for viral load. The results showed viral loads peaked at day 3 for both the mutants M-A43G and M-I36F/A43G, but slightly later, at day 5 pi, for wild-type and the M-I36F mutant (FIG. 11C). At day 3 or 5 pi, however, blood viral loads of M-I36F survivors and M-I36F/A43G were 3.4- or 14.7-fold and 4- or 7.6-fold lower, respectively, compared to that of wild-type-infected mice (FIG. 11C). Taken together these data indicate that the M-I36F/A43G and M-I36F viruses are rapidly cleared following infection. Sequence analyses of the entire M-I36F/A43G mutant genome collected from blood samples revealed no reversion to wild-type and no compensatory mutation (data not shown). This contrasts dramatically with the results of sequencing M-I36F viral genomes harvested from mice that did not survive the infection, which showed a reversion to the parental genotype (M-I36) (data not shown). Altogether, these results demonstrate that the M-I36F mutation strongly attenuates WNV in vivo and that the presence of the M-A43G mutation allows for stable retention of M-I36F attenuation without negative impact. Next, the inventors investigated the immunogenic profile of the M-I36F and M-I36F/A43G mutants in mice (FIG. 11, panels D and E). A single intraperitoneal injection of either M-I36F or M-I36F/A43G into adult BALB/c mice induced high levels of both WNV-specific IgG and neutralizing antibodies at day 27 post-infection (geometric mean titer=102.86 and 110 respectively, FIGS. 11D and 11E respectively). Induction of a remarkably robust neutralizing antibody response to WNV M-I36F and M-I36F/A43G in mice led the inventors to explore the protection afforded against a lethal challenge with the wild-type strain. Mice that had survived infection with M-I36F or M-I36F/A43G virus, or control mice injected with PBS, were infected with 1000 FFU of wild-type WNV. Not surprisingly, all but one control mouse developed symptoms upon intraperitoneal challenge and died from the infection around 8 days pi. Importantly, none of the mice that had been injected with a single dose of the M-I36F/A43G mutant virus exhibited symptoms after being challenged with wild-type WNV, and all survived the infection (FIG. 11F). Such protection was also conferred to mice that had survived M-I36F mutant infection when the mutant virus did not revert (FIG. 11F). These results demonstrate that the M-I36F and M-A43G mutation combination confers both full attenuation of WNV and full protection against wild-type WNV challenge in mice.

1. A live and attenuated flavivirus comprising a genome encoding a mutated M protein having an amino acid sequence that is at least 93% identical to the sequence of the wild type M protein of the flavivirus, wherein the amino acid at the position corresponding to amino acid position 36 of SEQ ID NO: 2 is replaced by an amino acid selected from the group consisting of phenylalanine, tryptophan and tyrosine; and wherein the amino acid at the position corresponding to amino acid position 43 of SEQ ID NO: 2 is replaced by glycine.

2. (canceled)

3. The live and attenuated flavivirus according to claim 1, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

4. The live and attenuated flavivirus according to claim 1, wherein the mutated flavivirus M protein has an amino acid sequence that consists of the amino acid sequence of the wild type M protein of the flavivirus, wherein the amino acid at the position corresponding to amino acid position 36 of SEQ ID NO: 2 is replaced by phenylalanine; and wherein the amino acid at the position corresponding to amino acid position 43 of SEQ ID NO: 2 is replaced by glycine.

5. The live and attenuated flavivirus according to claim 1, wherein the mutated M protein comprises an amino acid of sequence of from 8 to 49 amino acids, comprises an amino acid of sequence of from 8 to 15 amino acids, or comprises an amino acid sequence of from 8 to 25 amino acids of an amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84;
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by an amino acid selected from the group consisting of phenylalanine, tryptophan and tyrosine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine; or
wherein the amino acid at the position corresponding to amino acid position 36 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by phenylalanine; and wherein the amino acid at the position corresponding to amino acid position 43 of the amino acid sequence selected from the group consisting of SEQ ID NO: 19, SEQ ID NO: 20, SEQ ID NO: 21, SEQ ID NO: 22, SEQ ID NO: 84 and SEQ ID NO: 86, preferably of SEQ ID NO: 84, is replaced by glycine.

6. The live and attenuated flavivirus according to claim 1, which does not comprise a protein having the amino acid sequence of SEQ ID NO: 2.

7. (canceled)

8. A live and attenuated flavivirus selected from the group consisting of respectively Dengue virus 4 (DV4), West Nile virus (WNV), Japanese Encephalitis Virus (JEV), Zika virus (ZIKV) and Usutu virus (USUV), preferably is WNV or ZIKV, the genome of which encodes a mutated M protein, and wherein in said mutated M protein:
the amino acid at the position corresponding to amino acid position 36 of the amino acid sequence selected from the group consisting of respectively SEQ ID NO: 19 (DV4), SEQ ID NO: 20 (JEV), SEQ ID NO: 2 (WNV), SEQ ID NO: 21 (WNV), SEQ ID NO: 22 (ZIKV), SEQ ID NO: 84 (ZIKV) and SEQ ID NO: 86 (USUV) is replaced by an amino acid selected from the group consisting of phenylalanine, tryptophan and tyrosine; and wherein the amino acid at the position corresponding to amino acid position 43 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by glycine; or
the amino acid at the position corresponding to amino acid position 36 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by phenylalanine; and wherein the amino acid at the position corresponding to amino acid position 43 of the amino acid sequence selected from the group consisting of respectively SEQ ID NO: 19, SEQ ID NO: 20, SEQ ID NO: 2, SEQ ID NO: 21, SEQ ID NO: 22, SEQ ID NO: 84 and SEQ ID NO: 86, is replaced by glycine.

9-10. (canceled)

11. A nucleic acid, which is the RNA genomic nucleic acid of the live and attenuated flavivirus according to claim 1 or is a cDNA nucleic acid, the sequence of which is the retrotranscript of this RNA genomic nucleic acid.

12. (canceled)

13. A recombinant cell, which comprises the live and attenuated flavivirus of claim 1.

14. A cDNA clone of the live and attenuated flavivirus of claim 1.

15-16. (canceled)

17. An immunogenic composition, which comprises:
the live and attenuated flavivirus of claim 1 or viral particles thereof.

18. The live and attenuated flavivirus of claim 1, wherein the flavivirus is selected from the group consisting of Dengue virus 4 (DV4), Japanese Encephalitis Virus (JEV), Zika virus (ZIKV) and Usutu virus (USUV), preferably is ZIKV.

19. A live and attenuated West Nile Virus (WNV), comprising a genome encoding a mutated WNV M protein having an amino acid sequence that is at least 93% identical to the sequence of SEQ ID NO: 2; and wherein the amino acid at position 36 of SEQ ID NO: 2 is replaced by an amino acid selected from the group consisting of phenylalanine, tryptophan and tyrosine; and wherein the amino acid at position 43 of SEQ ID NO: 2 is replaced by glycine.

20. (canceled)

21. The live and attenuated WNV according to claim 19, wherein the amino acid at position 36 of SEQ ID NO: 2 is replaced by phenylalanine; and wherein the amino acid at position 43 of SEQ ID NO: 2 is replaced by glycine.

22. The live and attenuated WNV according to claim 19, wherein the mutated WNV M protein has an amino acid sequence that comprises or consists of SEQ ID NO: 4.

23. The live and attenuated WNV according to claim 19, which does not comprise a protein having the amino acid sequence of SEQ ID NO: 2.

24-26. (canceled)

27. A nucleic acid, which is the RNA genomic nucleic acid of the live and attenuated WNV according to claim 19 or is a cDNA nucleic acid, the sequence of which is the retrotranscript of this RNA genomic nucleic acid.

28. (canceled)

29. A recombinant cell, which comprises the live and attenuated WNV of claim 19.

30. A cDNA clone of the live and attenuated WNV of claim 19.

31-32. (canceled)

33. An immunogenic composition, which comprises:
the live and attenuated WNV of claim 19.

34. The immunogenic composition according to claim 17 for the treatment, prevention and/or protection against a flavivirus infection, in particular a WNV or ZIKV infection, in a mammalian host, preferably a human host.

35. (canceled)
